(19) United States
(12) Patent Application Publication
(10) Pub. No.: US 2020/0253143 A1
(43) Pub. Date: Aug. 13, 2020

(54) IPLANTS OF JUSTICIA AND THEIR USES

(71) Applicants: DANA-FARBER CANCER INSTITUTE, INC., Boston, MA (US); THE BRIGHAM AND WOMEN'S HOSPITAL, INC., Boston, MA (US)

(72) Inventor: Wilfred F. NGWA, Orlando, FL (US)

(21) Appl. No.: 16/649,278

(22) PCT Filed: Sep. 21, 2018
(86) PCT No.: PCT/US2018/052276

§ 371 Date: Mar. 20, 2020

Related U.S. Application Data
(60) Provisional application No. 62/561,488, filed on Sep. 21, 2017.

(51) Int. Cl. A01H 5/02; A01H 6/00; A23F 3/16

(52) U.S. Cl. CPC A01H 5/02; A23F 3/16; A01H 6/00

(57) ABSTRACT

A new species of Justicia plants preliminarily named Justicia sanguinis is disclosed. The present disclosure relates to the morphological and physiological characteristics of the newly discovered Justicia plants and their uses. The disclosure further relates to methods of making and drinking a beverage produced using a Justicia plant of the present invention or a part thereof.

### CROSS-REFERENCE TO RELATED APPLICATIONS

This application claims the benefit of priority to U.S. provisional application No. 62/561,488 filed on Sep. 21, 2017 which is hereby incorporated by reference in its entirety.

### FIELD

The present invention relates to the discovery and asexual reproduction of a distinct and new species of Justicia plant as well as to representative varieties of such plants. The present invention also relates to methods of producing, breeding and using such plants, such as for making tea beverages with certain health and other benefits.

### BACKGROUND

The following description includes information that may be useful in understanding the present disclosure. It is not an admission that any of the information provided herein is prior art or relevant to the presently claimed disclosures, or that any publication specifically or implicitly referenced is prior art.

Justicia is the largest genus of flowering plants in the family Acanthaceae. The Justicia genus has about 650 recognized species with hundreds of different plants possibly representing additional species. Justicia plants are typically found in pantropical and tropical climate areas. Plants of this genus are native in tropical to warm temperate regions, including Africa, the Americas, and India. Plants belonging to the Justicia genus are evergreen perennial plants. They are shrubs or subshrubs with strongly-veined leaves and lip-shaped corolla. For further information on this genus of plants, see, e.g., Austin, Daniel F. (2004), Florida Ethnobotany, CRC Press, p. 381, ISBN 978-0-8493-2332-4; and, RHS A-Z encyclopedia of garden plants, United Kingdom: Dorling Kindersley (2008), p. 1136, ISBN 1405332964.

Some Justicia species are cultivated for their ornamental value, while extracts of some species of Justicia are disclosed as being used for treating skin conditions, HIV, asthma, allergies, migraines and cancer.

There is a need to discover new species of Justicia that have the potential to positively impact the physical and psychological health of human beings; and, to develop new varieties of such species with improvements in their desirable plant traits.

### SUMMARY OF THE DISCLOSURE

The following embodiments and aspects thereof are described in conjunction with systems, tools and methods which are meant to be exemplary and illustrative, not limiting in scope.

In some embodiments, there is provided a novel Justicia plant species, preliminarily designated herein as Justicia sanguinis. One representative genotype of this new plant species is designated ‘Befu’. Tests are underway to confirm the initial saniguinis species designation of this new plant species discovered in a cultivated area and described herein. This invention thus relates to the Justicia plants as described herein, parts of the Justicia plants described herein, extracts of the Justicia plants described herein, and to plant cells of the Justicia plants described herein. The present invention also relates to plants or parts or extracts thereof consisting essentially of the phenotypic and morphological characteristics of the Justicia plants described herein, and/or having all the physiological and morphological characteristics of the Justicia plants described herein. The present invention also relates to plants having one or more or all of the characteristics of the Justicia plants described herein, but not limited to, as determined at the 5% significance level when grown in the same environmental conditions, including when grown side-by-side with a comparison or check plant of the same genus or species. The present invention also relates to Justicia plants having one or more of the physiological and morphological characteristics of the Justicia plants described herein including, but not limited to, as determined at the 5% significance level when grown in the same environmental conditions, including when grown side-by-side with a comparison or check plant of the same genus or species. The invention also relates to variants, mutants and trivial modifications of the Justicia plants of the present invention.

Plant parts of the Justicia plants of the present invention are also provided, such as leaf, stem, flower, fruit, seed, cell, pollen, stalk, roots, anther or ovule obtained from the Justicia plants. In some embodiments, the present invention provides leaves of the Justicia plants of the present invention. In other embodiments, the present invention provides stems of the Justicia plants of the present invention. Such leaf, a stem or parts thereof could be used as fresh products for consumption or in processes resulting in processed products such as fresh products comprising one or more parts of the Justicia plants of the present invention, such as prepared parts thereof, freeze dried or frozen parts thereof, dried and pulverized into powder and/or tea and the like, and such as a beverage comprising components obtained from one or more parts of the Justicia plants of the present invention. The harvested part or fresh and/or processed products comprise one or more parts of the Justicia plants of the present invention. The processed products might have undergone one or more processing steps such as, but not limited to cutting, washing, mixing, drying, freezing, pulverizing, making tea, producing beverage, etc. All such products are part of the present invention.

The plants and parts of the present invention include those that may be of an essentially derived variety as defined in section 41(3) of the Plant Variety Protection Act of The United States of America, e.g., a variety that is predominantly derived from the Justicia plants of the present invention or from a variety that i) is predominantly derived from the Justicia plants of the present invention, while retaining the expression of the essential characteristics that result from the genotype or combination of genotypes of the Justicia plants of the present invention; ii) is clearly distinguishable from the Justicia plants of the present invention; and iii) except for differences that result from the act of derivation, conforms to the initial variety in the expression of the essential characteristics that result from the genotype or combination of genotypes of the initial variety.

In some embodiments, the present invention provides regenerable cells. In some embodiments, the regenerable cells are for use in tissue culture of the Justicia plants of the present invention. In some embodiments, the tissue culture is capable of regenerating plants consisting essentially of the phenotypic and morphological characteristics of the Justicia plants of the present invention, and/or having all the phenotypic and morphological characteristics of the Justicia plants of the present invention, and/or having all the physiological and morphological characteristics of the Justicia plants of the present invention, and/or having all the characteristics of the Justicia plants of the present invention. In one embodiment, the regenerated plants have one or more or all of the characteristics of the Justicia plants described herein including but not limited to as determined at the 5% significance level when grown in the same environmental conditions as a comparison or check plant. In some embodiments, the whole plants regenerated from the tissue culture have one, more than one, or all of the physiological and morphological characteristics of the Justicia plants described herein including but not limited to as determined at the 5% significance level when grown in the same environmental conditions, including when grown side-by-side with a comparison or check plant. in some embodiments, the plant parts and cells used to produce such tissue cultures will be embryos, meristematic cells, seeds, callus, pollen, leaves, anthers, pistils, roots, root tips, stems, petioles, cotyledons, hypocotyls, ovaries, seed coat, stalks, endosperm, fruits, flowers, axillary buds or the like. Protoplasts produced from such tissue culture are also included in the present invention. The shoots, roots and whole plants regenerated from the tissue culture, as well as the fruit produced by said regenerated plants are also part of the invention. In some embodiments, the leaves, stems, and whole plants regenerated from the tissue culture are part of the invention.

The invention also discloses methods for vegetatively propagating a plant of the present invention. In some embodiments, the methods comprise collecting a part of the Justicia plants of the present invention and regenerating a plant from said part. In some embodiments, the part can be for example a stem cutting that is rooted into an appropriate medium according to techniques known by the one skilled in the art. Plants and plant parts thereof produced by such methods are also included in the present invention. In another aspect, the plants and parts thereof produced by such methods consist essentially of the phenotypic and morphological characteristics of the Justicia plants of the present invention, and/or having all the phenotypic and morphological characteristics of the Justicia plants of the present invention, and/or having all the physiological and morphological characteristics of the Justicia plants of the present invention, and/or having the characteristics of the Justicia plants of the present invention. In some embodiments, plants produced by such methods consist of one, more than one, or all phenotypic and morphological characteristics of the Justicia plants as described herein including but not limited to as determined at the 5% significance level when grown in the same environmental conditions, including when grown side-by-side with a comparison or check plant.

Furthermore, the invention teaches methods for producing plants and plant parts from the Justicia plants of the present invention. In some embodiments, the methods comprise growing the Justicia plants of the present invention to produce the Justicia plants and parts thereof including leaves and stems. In some embodiments, the methods further comprise harvesting the plants, plant parts, fruits and/or seeds. Such fruits and/or seeds are part of the present invention.

Also, this invention teaches methods for producing the Justicia plants of the present invention. In some embodiments, such Justicia plants are produced by crossing the Justicia plant with itself or another Justicia plant. In some embodiments, the second parent plant can be Justicia plant of the present invention, a plant of another variety of the Justicia plants of the present invention, or from other species in the Justicia genus. The Justicia hybrid plants and plant parts thereof produced by the method comprising crossing a Justicia plant of the present invention with a different Justicia plant and harvesting the resultant Justicia hybrid plants are included in the present invention, as are included the Justicia plants or parts or extracts thereof and seeds produced by growing and harvesting seeds from such Justicia hybrid plants.

Further included in the invention are methods for producing Justicia plants of the present invention and plant parts or extracts thereof. In some embodiments, such methods comprise planting, cultivating and harvesting Justicia plants of the present invention to produce the resultant plants, plant parts, extracts and seeds. Justicia plant seeds produced by such methods are also part of the invention.

In other embodiments, this invention relates to methods for producing a Justicia plant from a collection of seeds of the plants of the present invention. In some embodiments, the collection contains both seeds of Justicia plants selfed and/or Justicia plants crossed with another plant including Justicia plants of the present invention, another variety of the Justicia plants of the present invention, or other species in the Justicia genus. Such a representative collection of seeds include a commercial bag of seeds of the present invention. In some embodiments, said methods comprise planting the collection of seeds. When planted, the collection of seeds will produce Justicia plants.

This invention also relates to methods for producing other Justicia plants derived from Justicia plants of the present invention by the use of methods taught in this invention.

In some embodiments, such methods for producing a Justicia plant derived from the Justicia plants of the present invention comprise (a) crossing the Justicia of the present invention with a second Justicia plant to produce a progeny plant. In some embodiments, the methods further comprise (b) crossing the progeny plant derived from Justicia with itself or a second plant to produce a seed of progeny plant of subsequent generation; (c) growing the progeny plant of the subsequent generation from the seed (d) crossing the progeny plant of the subsequent generation with itself or a second plant, to produce a Justicia plant derived from the Justicia. In further embodiments, steps (b), (c) and/or (d) are repeated for at least 1, 2, 3, 4, 5, 6, 7, 8, or more generations to produce a Justicia plant derived from the Justicia plant. In some embodiments, within each crossing cycle, the second plant is the same plant as the second plant in the last crossing cycle. In some embodiments, within each crossing cycle, the second plant is different from the second plant in the last crossing cycle.

In some embodiments, such methods for producing a Justicia plant derived from Justicia of the present invention comprise (a) self-pollinating a Justicia plant of the present invention at least once to produce a progeny plant derived the Justicia plant of the present invention. In some embodiments, the methods further comprise (b) crossing the progeny plant derived from a Justicia of the present invention with itself or a second plant to produce a seed of progeny plant of subsequent generation; (c) growing the progeny plant of the subsequent generation from the seed (d) crossing the progeny plant of the subsequent generation with itself or a second plant, to produce a Justicia plant derived from the Justicia. In further embodiments, steps (b), (c) and/or (d) are repeated for at least 1, 2, 3, 4, 5, 6, 7, 8, or more generations to produce a Justicia plant derived from the Justicia plant. In some embodiments, within each crossing cycle, the second plant is the same plant as the second plant in the last crossing cycle. In some embodiments, within each crossing cycle, the second plant is different from the second plant in the last crossing cycle,

In some embodiments, the present invention provides methods of introducing or modifying one or more desired trait(s) a Justicia plant or parts thereof obtained from such methods. The desired trait(s) may be, but not exclusively, a single gene. In some embodiments, the gene is a dominant allele. In some embodiments, the gene is a partially dominant allele. In some embodiments, the gene is a recessive allele. In some embodiments, the gene or genes will confer such traits including, but not limited to male sterility, herbicide resistance, insect resistance, resistance for bacterial, fungal, mycoplasma or viral disease, enhanced plant quality such as improved drought or salt tolerance, water stress tolerance, improved standability, enhanced plant vigor, improved shelf life, delayed senescence or controlled ripening, enhanced nutritional quality such as increased sugar content or increased sweetness, increased texture, flavor and aroma, improved fruit length and/or size, protection or color, fruit shape, uniformity, length or diameter, refinement or depth, lodging resistance, yield and recovery. For the present invention and the skilled artisan, disease is understood to include, but not limited to fungal diseases, viral diseases, bacterial diseases, mycoplasm diseases, or other plant pathogenic diseases and a disease resistant plant will encompass a plant resistant to fungal, viral, bacterial, mycoplasm, and other plant pathogens. The gene or genes in Justicia plants the may be naturally occurring gene(s), mutant(s) or genes modified through New Breeding Techniques. In some embodiments, the method for introducing the desired trait(s) is a backcrossing process making use of a series of backcrosses to at least one of the parent lines of Justicia plants during which the desired trait(s) is maintained by selection. The single gene conversion plants that can be obtained by the methods are included in the present invention.

When dealing with a gene that has been modified, for example through New Breeding Techniques, the trait (genetic modification) could be directly modified into the newly developed line/cultivar such as at least one of the parent lines of Justicia plants. Alternatively, if the trait is not modified into each newly developed line/cultivar such as at least one of the parent lines of Justicia plants, another typical method used by breeders of ordinary skill in the art to incorporate the modified gene is to take a line already carrying the modified gene and to use such line as a donor line to transfer the modified gene into one or more of the parents of the newly developed Justicia plant.

The same would apply for a naturally occurring trait or one arising from spontaneous or induced mutations.

In some embodiments, the backcross breeding process of Justicia plant comprises (a) crossing one of the parental inbred lines of Justicia plants of the present invention with plants of another line that comprise the desired trait(s) to produce F1 progeny plants. In some embodiments, the process further comprises (b) selecting the F1 progeny plants that have the desired trait(s). In some embodiments, the process further comprises (c) crossing the selected F1 progeny plants with the parental lines of Justicia plants to produce backcross progeny plants. In some embodiments, the process further comprises (d) selecting for backcross progeny plants that have the desired trait(s) and physiological and morphological characteristics of the parental inbred line of Justicia plants to produce selected backcross progeny plants. In some embodiments, the process further comprises (e) repeating steps (c) and (d) one, two, three, four, five six, seven, eight, nine or more times in succession to produce selected, second, third, fourth, fifth, sixth, seventh, eighth, ninth or higher backcross progeny plants that have the desired trait(s) and consist essentially of the phenotypic and morphological characteristics of the parental lines of Justicia plants of the present invention, and/or have all the phenotypic and morphological characteristics of the parental lines of Justicia plants of the present invention, and/or have the desired trait(s) and the physiological and morphological characteristics of the parental lines of Justicia plants as described herein, including but not limited to, at a 5% significance level when grown in the same environmental conditions, including when grown side-by-side with an appropriate comparison or check plant. The Justicia plants or seed produced by the methods are also part of the invention, as are the Justicia plants that comprise the desired trait. Backcrossing breeding methods, well known to one skilled in the art of plant breeding will be further developed in subsequent parts of the specification.

Another embodiment of this invention includes methods of making a backcross of Justicia plants of the present invention so as to incorporate a mutant gene. In some embodiments, the method comprises crossing one of the parental lines of Justicia plants of the present invention with a donor plant comprising a mutant gene(s), a naturally occurring gene(s), or a gene and/or sequences modified through New Breeding Techniques conferring one or more desired trait to produce F1 progeny plants. In some embodiments, the method further comprises selecting an F1 progeny plant comprising the naturally occurring gene(s), mutant gene(s) or modified gene(s) and/or sequences conferring the one or more desired trait. In some embodiments, the method further comprises backcrossing the selected progeny plant with the parental lines of Justicia plants of the present invention. This method may further comprise the step of obtaining a molecular marker profile of the parental lines of Justicia plants and using the molecular marker profile to select for the progeny plant with the desired trait and the molecular marker profile the parental lines of Justicia plants. In some embodiments, this method further comprises crossing the backcross progeny plant containing the naturally occurring gene(s), the mutant gene(s) or the modified gene(s) and/or sequences conferring the one or more desired trait with the second parental lines of Justicia plants in order to produce the progeny Justicia plants comprising the naturally occurring gene(s), the mutant gene(s) or modified gene(s) and/or sequences conferring the one or more desired traits. The plants or parts thereof produced by such methods are also part of the present invention.

In some embodiments of the invention, the number of loci that may be backcrossed into the parental lines of Justicia plants is at least 1, 2, 3, 4, 5, or more. A single locus may contain several genes. A single locus conversion also allows for making one or more site specific changes to the plant genome, such as, without limitation, one or more nucleotide change, deletion, insertions, etc. In some embodiments, the single locus conversion is performed by genome editing, a.k.a. genome editing with engineered nucleases (GEEN). In some embodiments, the genome editing comprises using one or more engineered nucleases. In some embodiments, the engineered nucleases include, but are not limited to Zinc finger nucleases (ZFNs), Transcription Activator-Like Effector Nucleases (TALENs), the CRISPR/Cas system, and engineered meganuclease re-engineered horning endonucleases and endonucleases for DNA guided genome editing (Gao et al., Nature Biotechnology (2016), doi: 10.1038/nbt.3547). In some embodiments, the single locus conversion changes one or several nucleotides of the plant genome. Such genome editing techniques are some of the techniques now known by the person skilled in the art and herein are collectively referred to as “New Breeding Techniques”.

The invention further provides methods for developing Justicia plants using plant breeding techniques including but not limited to, recurrent selection, backcrossing, pedigree breeding, genomic selection, molecular marker (Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), and Simple Sequence Repeats (SSRs) which are also referred to as Microsatellites, Single Nucleotide Polymorphism (SNP), etc.) enhanced selection, genetic marker enhanced selection and transformation. The Justicia plants, and parts thereof produced by such breeding methods are also part of the invention.

The invention also relates to variants, mutants and trivial modifications of the Justicia plants of the present invention and parts and extracts thereof. Variants, mutants and trivial modifications of the Justicia plants and parts thereof can be generated by methods available to one skilled in the art, including but not limited to, mutagenesis (e.g., chemical mutagenesis, radiation mutagenesis, transposon mutagenesis, insertional mutagenesis, signature tagged mutagenesis, site-directed mutagenesis, and natural mutagenesis), knock-outs/knock-ins, antisense and RNA interference and other techniques such as the New Breeding Techniques.

This invention also is directed to methods for producing a Justicia plant by crossing a first parent Justicia plant of the present invention with a second parent Justicia plant wherein either the first or second parent plant is a Justicia plant. When crossed with another Justicia plant, a F1 Justicia seed is produced. Such methods of hybridization and self-pollination are well known to those skilled in the art of breeding.

Still further, this invention also is directed to methods for producing a Justicia derived from the Justicia of the instant invention by crossing a Justicia with a second Justicia plant. In some embodiments, the methods further comprise obtaining a progeny seed from the cross. In some embodiments, the methods further comprise growing the progeny seed, and possibly repeating the crossing and growing steps with a Justicia derived plant from 0 to 7 or more times. Thus, any such methods using a Justicia are part of this invention: selfing, backcrosses, hybrid production, crosses to populations, and the like. All plants produced using a Justicia of the present invention as a parent are within the scope of this invention, including plants derived from a Justicia. In some embodiments, such plants have one, more than one or all phenotypic and morphological characteristics of the a Justicia as described herein including but not limited to as determined at the 5% significance level when grown in the same environmental conditions, including when grown side-by-side with an appropriate comparison or check plant.

A Justicia plant of the present invention can be propagated vegetatively. A part of the plant, for example a stem and/or shoot tissue, is collected, and a new plant is obtained from the part. Such part typically comprises an apical meristem of the plant. The collected part is transferred to a medium allowing development of a plantlet, including for example rooting or development of shoots, or is grafted onto a Justicia plant or a rootstock prepared to support growth of shoot tissue. This is achieved using methods well-known in the art. Accordingly, in one embodiment, a method of vegetatively propagating a plant of the present invention comprises collecting a part of a plant according to the present invention, e.g. a stem and/or shoot tissue, and obtaining a plantlet from said part. In one embodiment, a method of vegetatively propagating a plant of the present invention comprises: a) collecting tissue of a plant of the present invention; b) rooting said proliferated stems and/or shoots to obtain rooted plantlets. In one embodiment, a method of vegetatively propagating a plant of the present invention comprises: a) collecting tissue of a plant of the present invention; b) cultivating said tissue to obtain proliferated stems and/or shoots; c) rooting said proliferated shoots to obtain rooted plantlets. In one embodiment, such method further comprises growing a plant from said plantlets. In one embodiment, seed is harvested from said plant.

The invention is also directed to the use of a Justicia plant of the present invention in a grafting process. In one embodiment, the Justicia plant is used as the scion while in another embodiment, the Justicia plant is used as a rootstock.

In one embodiment, the leaf and/or stem is processed into products such as beverage and/or tea that comprises Justicia plant of the present invention and/or parts thereof and/or extracts thereof. Such leaf, stem or parts thereof could be used as fresh products for consumption or in processes resulting in processed products such as fresh products comprising one or more parts of the Justicia plants, such as prepared parts thereof, freeze dried or frozen parts thereof, dried and pulverized into powder and/or tea and the like, and such as a beverage comprising components or extracts obtained from one or more parts of the Justicia plants.

In some embodiments, the present invention teaches a Alicia plant of the present invention, or a plant part thereof, or a plant cell thereof, wherein a representative sample of seed or tissue culture of said Justicia plant has been deposited with XXXX under XXXX No. ______.

In some embodiments, the present invention teaches, the Justicia plant, or a plant part thereof, or an extract thereof, or a plant cell thereof of, wherein the Justicia plant is the variety ‘Befu.’ In some embodiments, the present invention teaches a ‘Befu’ plant part, wherein the plant part is a leaf or a stem, or an extract from the plant or plant part of ‘Befu’. In some embodiments, the present invention teaches a Justicia plant having all of the characteristics of the variety ‘Befu’ as described herein when grown under the same environmental conditions, or a plant part or a plant cell thereof. In some embodiments, the present invention teaches a Justicia plant, or a plant part thereof, having all of the physiological and morphological characteristics of ‘Befu’.

In some embodiments, the present invention teaches a tissue culture of regenerable cells produced from the plant, plant part or plant cell, wherein a plant regenerated from the tissue culture has all of the characteristics of ‘Befu’ as described herein when grown under the same environmental conditions, In some embodiments, the present invention teaches a ‘Befu’ plant regenerated from the tissue culture, said plant having all the characteristics of ‘Befu’. In some embodiments, the present invention teaches a ‘Befu’ leaf produced from: 1) a plant deposited with XXXX under XXXX No; 2) a Justicia plant that is the variety ‘Befu’; 3) a plant having all the characteristics of ‘Befu’; 4) a plant having all of the physiological and morphological characteristics of ‘Befu’; and 5) a Justicia plant regenerated from the tissue culture of ‘Befu’.

In some embodiments, the present invention teaches a method for producing a ‘Befu’ leaf comprising a) growing a ‘Befu’ plant to produce a Justicia leaf, and b) harvesting said Justicia leaf. In some embodiments, the present invention teaches a ‘Befu’ leaf produced by a method comprising a) growing the Justicia plant to produce a Justicia leaf, and b) harvesting said Justicia leaf.

In some embodiments, the present invention teaches a method for producing a ‘Befu’ seed comprising crossing a ‘Befu’ plant with itself or a second, distinct Justicia plant. In some embodiments, the present invention teaches an F1 Justicia seed produced by the method for producing a Justicia seed comprising crossing a ‘Befu’ plant with itself or a second, distinct Justicia plant, and harvesting the resultant selfed or F1 seed.

In some embodiments, the present invention teaches a method for producing a Justicia seed. comprising self-pollinating a ‘Befu’ plant and harvesting the resultant Justicia seed. In some embodiments, the present invention teaches a ‘Befu’ seed produced by the method comprising self-pollinating the Justicia plant and harvesting the resultant Justicia seed.

In some embodiments, the present invention teaches a method of producing a Justicia plant derived from a ‘Befu’ plant, the method comprising (a) crossing the ‘Befu’ plant with a second Justicia plant to produce a progeny plant. The method further comprising the step of: (b) crossing the progeny plant derived from Justicia with itself or a second plant to produce a seed of progeny plant of subsequent generation; (c) growing the progeny plant of the subsequent generation from the seed (d) crossing the progeny plant of the subsequent generation with itself or a second plant, to produce a Justicia plant derived from the Justicia. The method further comprising the step of: (e) repeating steps (b) and/or (c) to produce a Justicia plant derived from the Justicia plant.

In some embodiments, the present invention teaches a Justicia plant comprising a single locus conversion and otherwise essentially all the characteristics of ‘Befu’ when grown in the same environmental conditions, In some embodiments, the present invention teaches that the single locus conversion confers said plant with herbicide resistance. in some embodiments, the present invention teaches the single locus conversion is an artificially mutated gene or nucleotide sequence. In some embodiments, the present invention teaches the single locus conversion is a gene that has been modified through the use of new breeding techniques.

In some embodiments, the present invention teaches a method of introducing a desired trait into ‘Befu’ comprising: (a) crossing a first ‘Befu’ plant with a second Justicia plant that comprises a desired trait to produce F1 progeny plants. The method further comprising the steps of: (b) selecting one or more progeny plants that have the desired trait to produce selected progeny plants; (c) crossing the selected progeny plants with the first ‘Befu’ plant so as to produce backcross progeny plants; (d) selecting for backcross progeny plants that have the desired trait and all of the physiological and morphological characteristics of the first Justicia plant when grown in the same environmental conditions to produce selected backcross progeny plants; and (e) repeating steps (c) and (d) three or more times in succession to produce selected fourth or higher backcross progeny plants that comprise the desired trait and all of the physiological and morphological characteristics of the first Justicia plant when grown in the same environmental conditions.

In some embodiments, the present invention teaches a beverage comprising an extract of a plant or plant part thereof of a Justicia plant of the present invention. In some embodiments, the present invention teaches a tea comprising an extract of a plant or plant part thereof of a Justicia plant of the present invention. In some embodiments the beverage is made using an extract from ‘Befu’.

In some embodiments, the present invention teaches an edible composition comprising an extract of a plant or plant part thereof of a Justicia plant of the present invention. In some embodiments, the present invention teaches that the plant part is leaf or a portion of a leaf. In some embodiments, the plant part is from a ‘Befu’ plant.

In some embodiments, the present invention teaches a method of preparing a beverage comprising placing a plant part of a Justicia plant of the present invention in contact with a liquid. In some embodiments, the present invention teaches that the plant part is a leaf or a portion of a leaf. In some embodiments, the present invention teaches that the leaf or portion of a leaf is partially or completely dried before placing it in the liquid. In some embodiments, the present invention teaches that the liquid is water. In some embodiments, the present invention teaches that the liquid is warm, hot or boiling when the leaf or portion of a leaf is placed into the liquid. In some embodiments, such a beverage is made using a plant part from ‘Befu’.

As set forth herein, the present invention teaches a new and distinct species of Justicia plants as described and illustrated in this invention. In some embodiments as set forth herein, the present invention teaches a new and distinct variety of Justicia named ‘Befu’ as described and illustrated in this invention.

### BRIEF DESCRIPTION OF THE DRAWINGS

FIGS. 1A and 1B provide a photograph of a ‘Befu’ plant grown in a cultivated area in Orlando, Fla., wherein the plant was asexually reproduced from a stem cutting from a parent plant.

### DETAILED DESCRIPTION

### Definitions

In the description and tables that follow, a number of terms are used. In order to provide a clear and consistent understanding of the specification and claims, including the scope to be given such terms, the following definitions are provided:

Allele. An allele is any of one or more alternative forms of a gene which relate to one trait or characteristic. In a diploid cell or organism, the two alleles of a given gene occupy corresponding loci on a pair of homologous chromosomes.

Backcrossing. Backcrossing is a process in which a breeder repeatedly crosses hybrid progeny back to one of the parents, for example, a first generation hybrid F₁ with one of the parental genotype of the F₁ hybrid.

Essentially all the physiological and morphological characteristics. A plant having essentially all the physiological and morphological characteristics means a plant having the physiological and morphological characteristics of the recurrent parent, except for the characteristics derived from the converted gene.

Immunity to disease(s) and or insect(s). A Justicia plant which is not subject to attack or infection by specific disease(s) and or insect(s) is considered immune.

Intermediate resistance to disease(s) and or insect(s). A Justicia plant that restricts the growth and development of specific disease(s) and or insect(s), but may exhibit a greater range of symptoms or damage compared to resistant plants. intermediate resistant plants will usually show less severe symptoms or damage than susceptible plant varieties when grown under similar environmental conditions and/or specific disease(s) and or insect(s) pressure, but may have heavy damage under heavy pressure. Intermediate resistant Justicia plants are not immune to the disease(s) and or insect(s).

Maturity (Date). Maturity refers to the stage when plants are of full size or optimum weight, and in marketable form or shape to be of commercial or economic value. In the region of best adaptability, maturity is the number of days from transplanting to optimal time for harvest.

New Breeding Techniques: New breeding techniques are said of various new technologies developed and/or used to create new characteristics in plants through genetic variation, the aim being targeted mutagenesis, targeted introduction of new genes or gene silencing (RdDM). Examples of such new breeding techniques are targeted sequence changes facilitated thru the use of Zinc finger nuclease (ZFN) technology (ZFN-1, ZFN-2 and ZFN-3, see U.S. Pat, No. 9,145,565, incorporated by reference in its entirety), Oligonucleotide directed mutagenesis (ODM), Cisgenesis and intragenesis, RNA-dependent DNA methylation (RdDM, which does not necessarily change nucleotide sequence but can change the biological activity of the sequence), Grafting (on GM rootstock), Reverse breeding, Agro-infiltration (agro-infiltration “sense stricto”, agro-inoculation, floral dip), Transcription Activator-Like Effector Nucleases (TALENs, see U.S. Pat. Nos. 8,586,363 and 9,181,535, incorporated by reference in their entireties), the CRISPR/Cas system (see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference), engineered meganuclease re-engineered homing endonucleases, DNA guided genome editing (Gao et al., Nature Biotechnology (2016), doi: 10.1038/nbt.3547, incorporated by reference in its entirety), and Synthetic genomics. A complete description of each of these techniques can be found in the report made by the Joint Research Center (JRC) Institute for Prospective Technological Studies of the European Commission in 2011 and titled “New plant breeding techniques—State-of-the-art and prospects for commercial development”, which is incorporated by reference in its entirety.

Plant adaptability. A plant having good plant adaptability means a plant that will perform well in different growing conditions and seasons.

Plant Cell. As used herein, the term “plant cell” includes plant cells whether isolated, in tissue culture or incorporated in a plant or plant part. In the present disclosure, this term refers to plant cells whether isolated in tissue culture or incorporated in a Justicia plant, a plant part thereof or an asexual clone thereof. Persons having skill in the art will appreciate that, unless otherwise noted, all references to a Justicia plant in the present disclosure can be read as referring to a plant cell from that plant. Therefore, embodiments described in the present disclosure which refer to a Justicia plant will also be understood to refer to a plant cell from said plant.

Plant Part. As used herein, the term “plant part” includes plant cells, plant protoplasts, plant cell tissue cultures from which Justicia plants can be regenerated, plant calli, plant clumps and plant cells that are intact in plants or parts of plants, such as embryos, pollen, ovules, flowers, seeds, rootstock, scions, stems, roots, anthers, pistils, root tips, leaves, meristematic cells, axillary buds, hypocotyls cotyledons, ovaries, seed coat endosperm and the like. In some embodiments, the plant part at least comprises at least one cell of said plant. In some embodiments, the plant part is further defined as a pollen, a meristem, a cell, or an ovule.

Quantitative Trait Loci (QTL) Quantitative trait loci refer to genetic loci that control to some degree numerically representable traits that are usually continuously distributed.

Regeneration. Regeneration refers to the development of a plant from tissue culture.

Resistance to disease(s) and or insect(s). A Justicia plant that restricts highly the growth and development of specific disease(s) and or insect(s) under normal disease(s) and or insect(s) attack pressure when compared to susceptible plants. These Justicia plants can exhibit some symptoms or damage under heavy disease(s) and or insect(s) pressure.

RHS. RHS refers to the Royal Horticultural Society of England which publishes an official botanical color chart quantitatively identifying colors according to a defined numbering system. The chart may be purchased from Royal Hort. Society Enterprise Ltd. RHS Garden; Wisley, Woking, Surrey GU236QB, UK.

Rootstock. A rootstock is the lower part of a plant capable of receiving a scion in a grafting process.

Scion. A scion is the higher part of a plant capable of being grafted onto a rootstock in a grafting process.

Single gene converted (conversion). Single gene converted (conversion) plants refer to plants which are developed by a plant breeding technique called backcrossing wherein essentially all of the desired morphological and physiological characteristics of a plant are recovered in addition to the single gene transferred into the plant via the backcrossing technique or via genetic engineering. A single gene converted plant can also be referred to a plant obtained though mutagenesis or through the use of some new breeding techniques, whereas the single gene converted plant has essentially all of the desired morphological and physiological characteristics of the original variety in addition to the single gene or nucleotide sequence muted or engineered through the new breeding techniques.

Susceptible to disease(s) and or insect(s). A Justicia plant that is susceptible to disease(s) and or insect(s) is defined as a Justicia plant that has the inability to restrict the growth and development of specific disease(s) and or insect(s). Plants that are susceptible will show damage when infected and are more likely to have heavy damage under moderate levels of specific disease(s) and or insect(s).

Tea beverage. Tea beverage means a composition produced by contacting/soaking (i.e. steeping) parts of a plant (e.g., leaves) in water for a period of time sufficient to extract components of the plant tissue. The tea beverages of the present disclosure are suitable for consumption by humans. Tea beverages, according to the present disclosure, include liquid concentrates of extracts from Justicia sanguinis plants (e.g., ‘Befu’ plants). Tea beverages of the present disclosure may be prepared in advance, and placed in a container (for example a bottle or can) as a ready-to-drink beverage. In some embodiments, a tea beverage may be also made by adding water (hot or cold) to fresh or dried leaves of a Justicia plant prior to consumption.

Tolerance to abiotic stresses. A Justicia plant that is tolerant to abiotic stresses has the ability to endure abiotic stress without serious consequences for growth, appearance and yield.

Uniformity. Uniformity, as used herein, describes the similarity between plants or plant characteristics which can be a described by qualitative or quantitative measurements.

Variety. A plant variety as used by one skilled in the art of plant breeding means a plant grouping within a single botanical taxon of the lowest known rank which can be defined by the expression of the characteristics resulting from a given genotype or combination of phenotypes, distinguished from any other plant grouping by the expression of at least one of the said characteristics and considered as a unit with regard to its suitability for being propagated unchanged (International convention for the protection of new varieties of plants). The term “cultivar” is used interchangeably with “variety” in this patent application.

### Justicia Plants

More commonly known plant species belonging to the Justicia genus include Justicia Americana, Justicia hrandegeeana, Justicia carnea, Justicia ovata, Justicia procumbens, Justicia pectoralis Jacq., Justicia gendarussa Buim. f., Justicia anselliana, and Justicia adhatoda.

Justicia americana (American water-willow) is an herbaceous, aquatic flowering plant in the Acanthus family native to eastern North America north to southern Ontario, and is known as the hardiest species in the genus. It is able to survive as far north as USDA Plant Zone 4, while other members of Justicia genes are largely tropical and subtropical. Justicia americana grows up to 40 cm in height from a creeping rhizome with opposite, sessile, linear or lanceolate, and slightly crenulated leaves and bicolored flowers born in opposite arrangement on spikes 3 cm in length coming off a peduncle 10 cm in length. The flowers are colored from white to pale lavender with the upper corolla lip pale violet or white, arching over the lower lip mottled in dark purple. The lateral lobes are unadorned or slightly blushed. The anthers are purplish-red rather than the usual yellow. The fruit of this plant is a small brown capsule. The flower blooms from May to October.

Justicia brandegeeana (formerly Beloperone guttata, commonly called shrimp plant or Mexican shrimp plant) is native to Mexico and also naturalized in Florida. Justicia brandegeeana grows to 1 m in height and 60-90 cm in width with oval green leaves 3-7.5 cm in length. The flowers are white, extending from red bracts like a shrimp, it is hardy to −4° C. but will often recover in the spring after freezing back in USDA Plant Zone 8a. Justicia carnea (formerly Jacobinia carnea, common names including Brazilian plume flower, flamingo flower, and jacobinia) is native to the Atlantic Forest ecoregions of eastern Brazil and South America in southern Brazil, Paraguay and northern Argentina. Justicia carnea is cultivated and sold as a decorative potted plant. It is hardy to −2° C. but will often recover in the spring after freezing back in USDA Plant Zone 8a.

Justicia procumbens (commonly known as Water Willow) is procumbent herb with angular sterns, swollen at nodes, small ovate leaves, small purple flowers in terminal spikes, inserted didynamous stamens, and shortly bilobed stigmas. Further, Justicia procumbens belonging to the Justicia genus of the Acanthaceae is an annual plant and is distributed in Korea, Japan, China, India, etc. Justicia procumbens has a height of about 30 cm, and its leaves are opposite and long oval in shape, 2-4 cm in length, and 1-2 cm in width. In addition, both ends of the leaf are pointed, and the edges of the leaf are elliptical or have a wave shape. The flower of the plant is light magenta in color, blooms in July to September, and bear fruit in September to October.

Varieties of some Justicia species are used as ornamental plants, including, e.g., J. pictifolia (e.g., cultivar ‘Zebra;’ U.S. Plant Patent No. 19,775); J. carnea, J. jacobina and J. aurea (collectively known as Brazilian plume flowers); and J. brandegeeana and J. whitelielda (collectively known as shrimp plants);

Botanical extracts of Justicia plants are used in methods and compositions for preventing, ameliorating or reducing a variety of human conditions and diseases, including (1) dermatological signs of aging (see, e.g., U.S. Patent Application Publication No. 2013/00552288 and WIPO Publication No. WO/2013/028266 (J. ventricosa)); (2) allergies (see, e.g., WIPO Publication No. WO/2016/060525); (3) HIV (see, e.g., U.S. Patent Application Publication No. 2014/0357584 and WIPO Publication No. WO/2013/019662 (J. gendarussa)); (4) skin lightening (see, e.g., WIPO Publication No. WO/2013/031403 (J. procumbens)); (5) bronchial asthma (see, e.g., WIPO Publication No. WO/2003/055558 (J. adhatoda)); (6) migraines (see, e.g., WIPO Publication No. WO/2007/048356 (J. pectoralis)); (7) for lowering cellular cholesterol and cholesteryl ester concentration (see, e.g., U.S. Pat. No. 6,365,411 (J. wynaadensis)); (8) cancer (see, e.g., U.S. Patent Application Publication No. 2004/0219226 and U.S. Pat. No. 7,005,146); and, (9) as a transglutaminase activator (see, e.g., U.S. Patent Application Publication No. 2015/0238404 and WIPO Publication No. WO/2014/034802 (J. procumbens)

### New Justicia Plants

The present disclosure relates to a new and distinct species of Justicia plants that botanically have not yet been given a scientific name, but is currently proposed by the inventor as Justicia sanguinis.

One new and distinct cultivar of Justicia sanguinis is the strain ‘Befu’. ‘Befu’ was initially discovered in a cultivated area on private land.

Asexual reproduction via stem cuttings was performed for the new cultivar ‘Befu’ in a cultivated area on private land in Orlando, Florida, U.S.A. Since that time, under careful observation, the unique characteristics of the new cultivar have been uniform, stable and reproduced true to type in successive generations of asexual reproduction.

Justicia sanguinis has important characteristics and traits, which distinguish the new and distinct cultivars of Justicia sanguinis from other existing known varieties of Justicia.

### Justicia Breeding

The goal of Justicia breeding is to develop new, unique and superior Justicia strains, varieties, cultivars and hybrids. The breeder initially selects and crosses two or more parental lines, followed by repeated selling and selection, producing many new genetic combinations. Another method used to develop new, unique and superior Justicia cultivar occurs when the breeder selects and crosses two or more parental lines followed by haploid induction and chromosome doubling that result in the development of dihaploid cultivars. The breeder can theoretically generate billions of different genetic combinations via crossing, selfing and mutations and the same is true for the utilization of the dihaploid breeding method.

Each year, the plant breeder selects the germplasm to advance to the next generation. This germplasm is grown under unique and different geographical, climatic and soil conditions, and further selections are then made, during and at the end of the growing season. The cultivars developed are unpredictable. This unpredictability is because the breeder's selection occurs in unique environments, with no control at the DNA level (using conventional breeding procedures or dihaploid breeding procedures), and with millions of different possible genetic combinations being generated. A breeder of ordinary skill in the art cannot predict the final resulting cultivars he develops, except possibly in a very gross and general fashion. This unpredictability results in the expenditure of large research monies to develop superior new Justicia cultivars.

The development of commercial Justicia cultivars requires the development and selection of Justicia plants, the crossing of these plants, and the evaluation of the crosses.

Pedigree breeding and recurrent selection breeding methods are used to develop cultivars from breeding populations. Breeding programs combine desirable traits from two or more cultivars or various broad-based sources into breeding pools from which cultivars are developed by selling and selection of desired phenotypes or through the dihaploid breeding method followed by the selection of desired phenotypes. The new cultivars are evaluated to determine which have commercial potential.

Choice of breeding or selection methods depends on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, recurrent selection, and backcross breeding.

### i Pedigree Selection

Pedigree breeding is used commonly for the improvement of self-pollinating crops or inbred lines of cross-pollinating crops. Two parents possessing favorable, complementary traits are crossed to produce an F₁. An F₂ population is produced by selting one or several F_{1S} or by intercrossing two F_{1S} (sib mating). The dihaploid breeding method could also be used. Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release of new cultivars. Similarly, the development of new cultivars through the dihaploid system requires the selection of the cultivars followed by two to five years of testing in replicated plots.

### ii Backcross Breeding

Backcross breeding has been used to transfer genes for a simply inherited, highly heritable trait into a desirable homozygous cultivar or inbred line which is the recurrent parent. The source of the trait to be transferred is called the donor parent, The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

When the term Justicia cultivar is used in the context of the present disclosure, this also includes any Justicia cultivar plant where one or more desired trait has been introduced through backcrossing methods, whether such trait is a naturally occurring one, a mutant or a gene or a nucleotide sequence modified by the use of New Breeding Techniques. Backcrossing methods can be used with the present disclosure to improve or introduce one or more characteristic into the Justicia cultivar of the present disclosure. The term “backcrossing” as used herein refers to the repeated crossing of a hybrid progeny back to the recurrent parent, i.e., backcrossing one, two, three, four, five, six, seven, eight, nine, or more times to the recurrent parent. The parental Justicia cultivar plant which contributes the gene or the genes for the desired characteristic is termed the nonrecurrent or donor parent. This terminology refers to the fact that the nonrecurrent parent is used one time in the backcross protocol and therefore does not recur. The parental Justicia cultivar to which the gene or genes from the nonrecurrent parent are transferred is known as the recurrent parent as it is used for several rounds in the backcrossing protocol.

In a typical backcross protocol, the original cultivar of interest (recurrent parent) is crossed to a second cultivar (nonrecurrent parent) that carries the gene or genes of interest to be transferred. The resulting progeny from this cross are then crossed again to the recurrent parent and the process is repeated until a Justicia plant is obtained wherein all the desired morphological and physiological characteristics of the recurrent parent are recovered in the converted plant, generally determined at a 5% significance level when grown in the same environmental conditions, in addition to the gene or genes transferred from the nonrecurrent parent. It has to be noted that some, one, two, three or more, self-pollination and growing of population might be included between two successive backcrosses. Indeed, an appropriate selection in the population produced by the self-pollination, i.e. selection for the desired trait and physiological and morphological characteristics of the recurrent parent might be equivalent to one, two or even three additional backcrosses in a continuous series without rigorous selection, saving then time, money and effort to the breeder. A non-limiting example of such a protocol would be the following: a) the first generation F1 produced by the cross of the recurrent parent A by the donor parent Bis backcrossed to parent A, b) selection is practiced for the plants having the desired trait of parent B, c) selected plant are self-pollinated to produce a population of plants where selection is practiced for the plants having the desired trait of parent B and physiological and morphological characteristics of parent A, d) the selected plants are backcrossed one, two, three, four, five, six, seven, eight, nine, or more times to parent A to produce selected backcross progeny plants comprising the desired trait of parent B and the physiological and morphological characteristics of parent A. Step (c) may or may not be repeated and included between the backcrosses of step (d).

The selection of a suitable recurrent parent is an important step for a successful backcrossing procedure. The goal of a backcross protocol is to alter or substitute one or more trait(s) or characteristic(s) in the original inbred parental line in order to find it then in the hybrid made thereof. To accomplish this, a gene or genes of the recurrent inbred is modified or substituted with the desired gene or genes from the nonrecurrent parent, while retaining essentially all of the rest of the desired genetic, and therefore the desired physiological and morphological, constitution of the original inbred. The choice of the particular nonrecurrent parent will depend on the purpose of the backcross; one of the major purposes is to add some commercially desirable, agronomically important trait(s) to the plant. The exact backcrossing protocol will depend on the characteristic(s) or trait(s) being altered to determine an appropriate testing protocol. Although backcrossing methods are simplified when the characteristic being transferred is a single gene and dominant allele, multiple genes and recessive allele(s) may also be transferred and therefore, backcross breeding is by no means restricted to character(s) governed by one or a few genes. In fact the number of genes might be less important that the identification of the character(s) in the segregating population. In this instance it may then be necessary to introduce a test of the progeny to determine if the desired characteristic(s) has been successfully transferred. Such tests encompass visual inspection, simple crossing, but also follow up of the characteristic(s) through genetically associated markers and molecular assisted breeding tools. For example, selection of progeny containing the transferred trait is done by direct selection, visual inspection for a trait associated with a dominant allele, while the selection of progeny for a trait that is transferred via a recessive allele, such as the waxy starch characteristic in corn, require selfing the progeny to determine which plant carry the recessive allele(s).

Many single gene traits have been identified that are not regularly selected for in the development of a new parental inbred of a hybrid lettuce plant according to the disclosure but that can be improved by backcrossing techniques. These genes are generally inherited through the nucleus.

In 1981, the backcross method of breeding counted for 17% of the total breeding effort for inbred line development in the United States, accordingly to, Hallauer, A. R. et al. (1988) “Corn Breeding” Corn and Corn Improvement, No. 18, pp. 463-481.

The backcross breeding method provides a precise way of improving varieties that excel in a large number of attributes but are deficient in a few characteristics. (Page 150 of the Pr. RM. Allard's 1960 book, published by John Wiley & Sons, Inc, Principles of Plant Breeding). The method makes use of a series of backcrosses to the variety to be improved during which the character or the characters in which improvement is sought is maintained by selection. At the end of the backcrossing the gene or genes being transferred unlike all other genes, will be heterozygous. Selfing after the last backcross produces homozygosity for this gene pair(s) and, coupled with selection, will result in a parental line of a hybrid variety with exactly the adaptation, yielding ability and quality characteristics of the recurrent parent but superior to that parent in the particular characteristic(s) for which the improvement program was undertaken. Therefore, this method provides the plant breeder with a high degree of genetic control of his work.

The method is scientifically exact because the morphological and agricultural features of the improved variety could be described in advance and because the same variety could, if it were desired, be bred a second time by retracing the same steps (Briggs, “Breeding wheats resistant to bunt by the backcross method”, 1930 Jour. Amer. Soc. Agron., 22: 289-244).

Backcrossing is a powerful mechanism for achieving homozygosity and any population obtained by backcrossing must rapidly converge on the genotype of the recurrent parent. When backcrossing is made the basis of a plant breeding program, the genotype of the recurrent parent will be modified only with regards to genes being transferred, which are maintained in the population by selection.

Successful backcrosses are, for example, the transfer of stem rust resistance from ‘Hope’ wheat to ‘Bart wheat’ and even pursuing the backcrosses with the transfer of bunt resistance to create ‘Bart 38’, having both resistances. Also highlighted by Allard is the successful transfer of mildew, leaf spot and wilt resistances in California. Common alfalfa to create ‘Caliverde’. This new ‘Caliverde’ variety produced through the backcross process is indistinguishable from California Common except for its resistance to the three named diseases.

One of the advantages of the backcross method is that the breeding program can be carried out in almost every environment that will allow the development of the character being transferred.

The backcross technique is not only desirable when breeding for disease resistance but also for the adjustment of morphological characters, color characteristics and simply inherited quantitative characters such as earliness, plant height and seed size and shape.

### iii Single-Seed Descent and Multiple Seed Procedures

The single-seed descent procedure in the strict sense refers to planting a segregating population, harvesting a sample of one seed per plant, and using the one-seed sample to plant the next generation. When the population has been advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generation advance is completed.

In a multiple-seed procedure, breeders commonly harvest one or more flower containing seed from each plant in a population and blend them together to form a bulk seed lot. Part of the bulked seed is used to plant the next generation and part is put in reserve. The procedure has been referred to as modified single-seed descent or the bulk technique.

The multiple-seed procedure has been used to save labor at harvest. It is considerably faster than removing one seed from each flower by hand for the single seed procedure. The multiple-seed procedure also makes it possible to plant the same number of seeds of a population each generation of inbreeding. Enough seeds are harvested to make up for those plants that did not germinate or produce seed.

Descriptions of other breeding methods that are commonly used for different traits and crops can be found in one of several reference books (e.g., R. W. Allard, 1960, Principles of Plant Breeding, John Wiley and Son, pp. 115-161; N.W. Simmonds, 1979, Principles of Crop Improvement, Longman Group Limited; W. R. Fehr, 1987, Principles of Crop Development, Macmillan Publishing Co.; N. F. Jensen, 1988, Plant Breeding Methodology, John Wiley & Sons).

### iii Open-Pollinated Populations

The improvement of open-pollinated populations of such crops as rye, maize and sugar beets, herbage grasses, legumes such as alfalfa and clover, and tropical tree crops such as cacao, coconuts, oil palm and some nibber, depends essentially upon changing gene-frequencies towards fixation of favorable alleles while maintaining a high (but far from maximal) degree of heterozygosity.

Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes to flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement.

First, there is the situation in which a population is changed en masse by a chosen selection procedure. The outcome is an improved population that is indefinitely propagable by random-mating within itself in isolation.

Second, the synthetic variety attains the same end result as population improvement, but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Pant Breeding, John Wiley & Sons, Inc. (1960); Simmonds, Principles of Crop Improvement, Longman Group Limited (1979); Hanauer and Miranda, Quantitative Genetics in Maize Breeding, Iowa State University Press (1981); and, Jensen, Plant Breeding Methodology, John Wiley & Sons, Inc. (1988),

### A) Mass Selection

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued. In mass selection, desirable individual plants are chosen, harvested, and the seed composited without progeny testing to produce the following generation. Since selection is based on the maternal parent only, and there is no control over pollination, mass selection amounts to a form of random mating with selection. As stated above, the purpose of mass selection is to increase the proportion of superior genotypes in the population.

### B) Synthetics

A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or toperosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or more cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enters a synthetic varies widely. In practice, numbers of parental lines range from 10 to several hundred, with 100-200 being the average. Broad based synthetics formed from 100 or more clones would be expected to be more stable during seed multiplication than narrow based synthetics.

iv. Hybrids

A hybrid is an individual plant resulting from a cross between parents of differing genotypes. Commercial hybrids are now used extensively in many crops, including corn (maize), sorghum, sugarbeet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

Once the inbreds that give the best hybrid performance have been identified, the hybrid seed can be reproduced indefinitely as long as the homogeneity of the inbred parent is maintained. A single-cross hybrid is produced when two inbred lines are crossed to produce the F1 progeny. A double-cross hybrid is produced from four inbred lines crossed in pairs (AxB and CxD) and then the two F1 hybrids are crossed again (AxB)×(CxD). Much of the hybrid vigor and uniformity exhibited by F1 hybrids is lost in the next generation (F2). Consequently, seed from F2 hybrid varieties is not used for planting stock.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8:161-176, In Hybridization of Crop Plants.

v. Bulk Segregation Analysis (BSA)

BSA, a.k.a. bulked segregation analysis, or bulk segregant analysis, is a method described by Michelmore et al, (Michelmore et al., 1991, identification of markers linked to disease-resistance genes by bulked segregant analysis: a rapid method to detect markers in specific genomic regions by using segregating populations. Proceedings of the National Academy of Sciences, USA, 99:9828-9832) and Quarrie et al. (Quarrie et al., 1999, Journal of Experimental Botany, 50(337):1299-1306).

For BSA of a trait of interest, parental lines with certain different phenotypes are chosen and crossed to generate F2, doubled haploid or recombinant inbred populations with QTL analysis. The population is then phenotyped to identify individual plants or lines having high or low expression of the trait. Two DNA bulks are prepared, one from the individuals having one phenotype (e.g., resistant to virus), and the other from the individuals having reversed phenotype (e.g., susceptible to virus), and analyzed for allele frequency with molecular markers. Only a few individuals are required in each bulk (e.g., 10 plants each) if the markers are dominant (e.g., RAPDs). More individuals are needed when markers are co-dominant (e.g., RFLPs, SNPs or SSRs). Markers linked to the phenotype can be identified and used for breeding or QTL mapping.

vi. Hand-Pollination Method

Hand pollination describes the crossing of plants via the deliberate fertilization of female ovules with pollen from a desired male parent plant. In some embodiments the donor or recipient female parent and the donor or recipient male parent line are planted in the same field. In some embodiments the donor or recipient female parent and the donor or recipient male parent line are planted in the same greenhouse. The inbred male parent can be planted earlier than the female parent to ensure adequate pollen supply at the pollination time. In some embodiments, the male parent and female parent can be planted at a ratio of 1 male parent to 4-10 female parents. Pollination is started when the female parent flower is ready to be fertilized. Female flower buds that are ready to open in the following days are identified, covered with paper cups or small paper bags that prevent bee or any other insect from visiting the female flowers, and marked with any kind of material that can be easily seen the next morning. The male flowers of the male parent are collected in the early morning before they are open and visited by pollinating insects. The covered. female flowers of the female parent, which have opened, are un-covered and pollinated with the collected fresh male flowers of the male parent, starting as soon as the male flower sheds pollen. The pollinated female flowers are again covered after pollination to prevent bees and any other insects visit. The pollinated female flowers are also marked. The marked flowers are harvested. In some embodiments, the male pollen used for fertilization has been previously collected and stored.

vii. Bee-Pollination Method

Using the bee-pollination method, the parent plants are usually planted within close proximity. In some embodiments more female plants are planted to allow for a greater production of seed. Insects are placed in the field or greenhouses for transfer of pollen from the male parent to the female flowers of the female parent.

viii. Targeting Induced Local Lesions in Genomes (TILLING)

Breeding schemes of the present application can include crosses with TILLING® plant cultivars. TILLING® is a method in molecular biology that allows directed identification of mutations in a specific gene. TILLING® was introduced in 2000, using the model plant Arahidopsis thaliana. TILLING® has since been used as a reverse genetics method in other organisms such as zebrafish, corn, wheat, rice, soybean, tomato and lettuce.

The method combines a standard and efficient technique of mutagenesis with a chemical mutagen (e.g., Ethyl methanesulfonate (EMS)) with a sensitive DNA screening-technique that identifies single base mutations (also called point mutations) in a target gene. EcoTILLING is a method that uses TILLING® techniques to look for natural mutations in individuals, usually for population genetics analysis (see Comai, et al., 2003 The Plant Journal 37, 778-786; Gilchrist et al. 2006 Mol. Ecol. 15, 1367-1378; Mejlhede et al. 2006 Plant Breeding 125. 461-467; Nieto et al. 2007 BMC Plant Biology 7, 34-42, each of which is incorporated by reference hereby for all purposes). DEcoTILLING is a modification of TILLING® and EcoTILLING which uses an inexpensive method to identify fragments (Garvin et al., 2007, DEco-TILLING: An inexpensive method for SNP discovery that reduces ascertainment bias. Molecular Ecology Notes 7, 735-746).

The TILLING® method relies on the formation of heteroduplexes that are formed when multiple alleles (which could be from a heterozygote or a pool of multiple homozygotes and heterozygotes) are amplified in a PCR, heated, and then slowly cooled. As DNA bases are not pairing at the mismatch of the two DNA strands (the induced mutation in TIILLING® or the natural mutation or SNP in EcoTILLING), they provoke shape change in the double strand DNA fragment which is then cleaved by single stranded nucleases. The products are then separated by size on several different platforms.

More detailed description on methods and compositions on TILLING® can be found in U.S. Pat. No. 5,994,075, US 2004/0053236 A1, WO 2005/055704, and WO 2005/048692, each of which is hereby incorporated by reference for all purposes.

Thus in some embodiments, the breeding methods of the present disclosure include breeding with one or more TILLING plant lines with one or more identified mutations.

viii Mutation Breeding

Mutation breeding is another method of introducing new variation and subsequent traits into plants. Mutations that occur spontaneously or are artificially induced can be useful sources of variability for a plant breeder. The goal of artificial mutagenesis is to increase the rate of mutation for a desired characteristic. Mutation rates can be increased by many different means or mutating agents including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in W. R. Fehr, 1993, Principles of Cultivar Development, Macmillan Publishing Co.

New breeding techniques such as the ones involving the uses of Zinc Finger Nucleases or oligonucleotide directed mutagenesis shall also be used to generate genetic variability and introduce new traits into varieties.

### .ix Double Haploids and Chromosome Doubling

One way to obtain homozygous plants without the need to cross two parental lines followed by a long selection of the segregating progeny, and/or multiple backcrossings is to produce haploids and then double the chromosomes to form doubled haploids. Haploid plants can occur spontaneously, or may be artificially induced via chemical treatments or by crossing plants with inducer lines (Seymour et al. 2012, PNAS vol 109, pg 4227-4232; Zhang et al., 2008 Plant Cell Rep. December 27(12) 1851-60). The production of haploid progeny can occur via a variety of mechanisms which can affect the distribution of chromosomes during gamete formation. The chromosome complements of haploids sometimes double spontaneously to produce homozygous doubled haploids (DHs). Mixoploids, which are plants which contain cells having different ploidies, can sometimes arise and may represent plants that are undergoing chromosome doubling so as to spontaneously produce doubled haploid tissues, organs, shoots, floral parts or plants. Another common technique is to induce the formation of double haploid plants with a chromosome doubling treatment such as colchicine (El-Hennawy et al., 2011 Vol 56, issue 2 pg 63-72; Doubled Haploid Production in Crop Plants 2003 edited by Maluszynski ISBN 1-4020-1544-5). The production of doubled haploid plants yields highly uniform cultivars and is especially, desirable as an alternative to sexual inbreeding of longer-generation crops. By producing doubled haploid progeny, the number of possible gene combinations for inherited traits is more manageable. Thus, an efficient doubled haploid technology can significantly reduce the time and the cost of inbred and cultivar development.

x. Protoplaast Fusion

In another method for breeding plants, protoplast fusion can also be used for the transfer of trait-conferring genomic material from a donor plant to a recipient plant. Protoplast fusion is an induced or spontaneous union, such as a somatic hybridization, between two or more protoplasts (cells of which the cell walls are removed by enzymatic treatment) to produce a single bi- or multi-nucleate cell. The fused cell that may even be obtained with plant species that cannot be interbred in nature is tissue cultured into a hybrid plant exhibiting the desirable combination of traits.

xi. Embryo Rescue

Alternatively, embryo rescue may be employed in the transfer of resistance-conferring genotnic material from a donor plant to a recipient plant. Embryo rescue can be used as a procedure to isolate embryo's from crosses wherein plants fail to produce viable seed. In this process, the fertilized ovary or immature seed of a plant is tissue cultured to create new plants (see Pierik, 1999, In vitro culture of higher plants, Springer, ISBN 079235267x, 9780792352679, which is incorporated herein by reference in its entirety).

### Breeding Evaluation

Each breeding program can include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested per se and in hybrid combination and compared to appropriate standards in environments representative of the commercial target area(s). The best lines are candidates for use as parents in new commercial cultivars; those still deficient in a few traits may be used as parents to produce new populations for further selection.

In one embodiment, the plants are selected on the basis of one or more phenotypic traits. Skilled persons will readily appreciate that such traits include any observable characteristic of the plant, including for example growth rate, height, weight, color, taste, smell, changes in the production of one or more compounds by the plant (including for example, metabolites, proteins, drugs, carbohydrates, oils, and any other compounds).

A most difficult task is the identification of individuals that are genetically superior, because for most traits the true genotypic value is masked by other confounding plant traits or environmental factors. One method of identifying a superior plant is to observe its performance relative to other experimental plants and to a widely grown standard cultivar. If a single observation is inconclusive, replicated observations provide a better estimate of its genetic worth,

Proper testing should detect any major faults and establish the level of superiority or improvement over current cultivars. In addition to showing superior performance, there must be a demand for a new cultivar that is compatible with industry standards or which creates a new market. The introduction of a new cultivar will incur additional costs to the seed producer, the grower, processor and consumer; for special advertising and marketing, altered seed and commercial production practices, and new product utilization. The testing preceding release of a new cultivar should take into consideration research and development costs as well as technical superiority of the final cultivar. For seed.-propagated cultivars, it must be feasible to produce seed easily and economically.

It should be appreciated that in certain embodiments, plants may be selected based on the absence, suppression or inhibition of a certain feature or trait (such as an undesirable feature or trait) as opposed to the presence of a certain feature or trait (such as a desirable feature or trait).

Selecting plants based on genotypic information is also envisaged (for example, including the pattern of plant gene expression, genotype, or presence of genetic markers). Where the presence of one or more genetic marker is assessed, the one or more marker may already be known and/or associated with a particular characteristic of a plant; for example, a marker or markers may be associated with an increased growth rate or metabolite profile. This information could be used in combination with assessment based on other characteristics in a method of the disclosure to select for a combination of different plant characteristics that may be desirable. Such techniques may be used to identify novel quantitative trait loci (QTLs). By way of example, plants may be selected based on growth rate, size (including but not limited to weight, height, leaf size, stern size, branching pattern, or the size of any part of the plant), general health, survival, tolerance to adverse physical environments and/or any other characteristic, as described herein before.

Further non-limiting examples include selecting plants based on: speed of seed germination; quantity of biomass produced; increased root, and/or leaf/shoot growth that leads to an increased yield (herbage or grain or fiber or oil, or fruit or leaves) or biomass production; effects on plant growth that results in an increased seed yield for a crop; effects on plant growth which result in an increased yield; effects on plant growth that lead to an increased resistance or tolerance to disease including fungal, viral or bacterial diseases, to mycoplasma or to pests such as insects, mites or nematodes in which damage is measured by decreased foliar symptoms such as the incidence of bacterial or fungal lesions, or area of damaged foliage or reduction in the numbers of nematode cysts or galls on plant roots, or improvements in plant yield in the presence of such plant pests and diseases; effects on plant growth that lead to increased metabolite yields; effects on plant growth that lead to improved aesthetic appeal which may be particularly important in plants grown for their form, color or taste, for example the color intensity of Justicia leaves, or the taste of said leaves.

### Molecular Breeding Evaluation Techniques

Selection of plants based on phenotypic or genotypic information may be performed using techniques such as, but not limited to: high through-put screening of chemical components of plant origin, sequencing techniques including high through-put sequencing of genetic material, differential display techniques (including DDRT-PCR, and. DD-PCR), nucleic acid microarray techniques, RNA-seq (transcriptome sequencing), qRTPCR (quantitative real time PCR).

In one embodiment, the evaluating step of a plant breeding program involves the identification of desirable traits in progeny plants. Progeny plants can be grown in, or exposed to conditions designed to emphasize a particular trait (e.g. drought conditions for drought tolerance, lower temperatures for freezing tolerant traits). Progeny plants with the highest scores for a particular trait may be used for subsequent breeding steps.

In some embodiments, plants selected from the evaluation step can exhibit a 1%, 5%, 10%, 15%, 20%. 25%, 30%, 35%, 40%, 45%, 50%, 55%. 60%, 65%, 70%, 75%, 80%, 85%, 90%, 95%, 100%, 120% or more improvement in a particular plant trait compared to a control plant.

In other embodiments, the evaluating step of plant breeding comprises one or more molecular biological tests for genes or other markers. For example, the molecular biological test can involve probe hybridization and/or amplification of nucleic acid (e.g., measuring nucleic acid density by Northern or Southern hybridization, PCR) and/or immunological detection (e.g., measuring protein density, such as precipitation and agglutination tests, ELISA (e.g., Lateral Flow test or DAS-ELISA), Western blot, immune labeling, immunosorbent electron microscopy (ISEM), and/or dot blot).

The procedure to perform a nucleic acid hybridization, an amplification of nucleic acid (e.g., PCR, RT-PCR) or an immunological detection (e.g., precipitation and agglutination tests, ELISA (e.g., Lateral Flow test or DAS-ELISA), Western blot, RIA, immunogold or immunofluorescent labeling, immunosorbent electron microscopy (ISEM), and/or dot blot tests) are performed as described elsewhere herein and well-known by one skilled in the art.

In one embodiment, the evaluating step comprises PCR (semi-quantitative or quantitative), wherein primers are used to amplify one or more nucleic acid sequences of a desirable gene, or a nucleic acid associated with said gene or QTL or a desirable trait (e.g., a co-segregating nucleic acid, or other marker).

In another embodiment, the evaluating step comprises immunological detection (e.g., precipitation and agglutination tests, ELISA (e.g., Lateral Flow test or DAS-ELISA), Western blot, RIA, immuno labeling (gold, fluorescent, or other detectable marker), immunosorbent electron microscopy (ISEM), and/or dot blot), wherein one or more gene or marker-specific antibodies are used to detect one or more desirable proteins. In one embodiment, said specific antibody is selected. from the group consisting of polyclonal antibodies, monoclonal antibodies, antibody fragments, and combination thereof.

Reverse Transcription Polymerase Chain Reaction (RT-PCR) can be utilized in the present disclosure to determine expression of a gene to assist during the selection step of a breeding scheme. It is a variant of polymerase chain reaction (PCR), a laboratory technique commonly used in molecular biology to generate many copies of a DNA sequence, a process termed “amplification”. In RT-PCR, however, RNA strand is first reverse transcribed into its DNA complement (complementary DNA, or cDNA) using the enzyme reverse transcriptase, and the resulting cDNA is amplified using traditional or real-time PCR.

RT-PCR utilizes a pair of primers, which are complementary to a defined sequence on each of the two strands of the mRNA. These primers are then extended by a DNA polymerase and a copy of the strand is made after each cycle, leading to logarithmic amplification.

RT-PCR includes three major steps. The first step is the reverse transcription (RT) where RNA is reverse transcribed to cDNA using a reverse transcriptase and primers. This step is very important in order to allow the performance of PCR since DNA polymerase can act only on DNA templates. The RI step can be performed either in the same tube with PCR (one-step PCR) or in a separate one (two-step PCR) using a temperature between 40° C. and 50° C., depending on the properties of the reverse transcriptase used.

The next step involves the denaturation of the dsDNA at 95° C., so that the two strands separate and the primers can bind again at lower temperatures and begin a new chain reaction. Then, the temperature is decreased until it reaches the annealing temperature which can vary depending on the set of primers used, their concentration, the probe and its concentration (if used), and the cation concentration. The main consideration, of course, when choosing the optimal annealing temperature is the melting temperature (Tm) of the primers and probes (if used). The annealing temperature chosen for a PCR depends directly on length and composition of the primers. This is the result of the difference of hydrogen bonds between A-T (2 bonds) and G-C (3 bonds). An annealing temperature about 5 degrees below the lowest Tin of the pair of primers is usually used.

The final step of PCR amplification is the DNA extension from the primers which is done by the thermostable Taq DNA polymerase usually at 72° C., which is the optimal temperature for the polymerase to work. The length of the incubation at each temperature, the temperature alterations and the number of cycles are controlled by a programmable thermal cycler. The analysis of the PCR products depends on the type of PCR applied. If a conventional PCR is used, the PCR product is detected using for example agarose gel electrophoresis or other polymer gel like polyacrylamide gels and ethidium bromide (or other nucleic acid staining).

Conventional RT-PCR is a time-consuming technique with important limitations when compared to real time PCR techniques. Furthermore, the specificity of the assay is mainly determined by the primers, which can give false-positive results. However, the most important issue concerning conventional RT-PCR is the fact that it is a semi or even a low quantitative technique, where the amplicon can be visualized only after the amplification ends.

Real time RT-PCR provides a method where the amplicons can be visualized as the amplification progresses using a fluorescent reporter molecule. There are three major kinds of fluorescent reporters used in real time RT-PCR, general nonspecific DNA Binding Dyes such as SYBR Green TaqMan Probes and Molecular Beacons (including Scorpions).

The real time PCR thermal cycler has a fluorescence detection threshold, below which it cannot discriminate the difference between amplification generated signal and background noise. On the other hand, the fluorescence increases as the amplification progresses and the instrument performs data acquisition during the annealing step of each cycle. The number of amplicons will reach the detection baseline after a specific cycle, which depends on the initial concentration of the target DNA sequence. The cycle at which the instrument can discriminate the amplification generated fluorescence from the background noise is called the threshold cycle (Ct). The higher is the initial DNA concentration, the lower its Ct will be.

Other forms of nucleic acid detection can include next generation sequencing methods such as DNA SEQ or RNA SEQ using any known sequencing platform including, but not limited to: Roche 454, Solexa Genome Analyzer, AB SOLiD, Illumina GA/HiSeq, Ion PGM, Mi Seq, among others (Liu et al,. 2012 Journal of Biomedicine and Biotechnology Volume 2012 ID 251364; Franca et al., 2002 Quarterly Reviews of Biophysics 35 pg. 169-200; Mardis 2008 Genomics and :Human Genetics vol 9 pg 387-402).

In other embodiments, nucleic acids may be detected with other high throughput hybridization technologies including microarrays, gene chips, LNA probes, nanoStrings, and fluorescence polarization detection among others.

In some embodiments, detection of markers can be achieved at an early stage of plant growth by harvesting a small tissue sample (e.g., branch, or leaf disk). This approach is preferable when working with large populations as it allows breeders to weed out undesirable progeny at an early stage and conserve growth space and resources for progeny which show more promise. In some embodiments the detection of markers is automated, such that the detection and storage of marker data is handled by a machine. Recent advances in robotics have also led to full service analysis tools capable of handling nucleic acid/protein marker extractions, detection, storage and analysis.

### Quantitative Trait Loci

Breeding schemes of the present application can include crosses between donor and recipient plants. In some embodiments said donor plants contain a gene or genes of interest which may confer the plant with a desirable phenotype. The recipient line can be an elite line or cultivar having certain favorite traits such for commercial production. In one embodiment, the elite line may contain other genes that also impart said line with the desired phenotype. When crossed together, the donor and recipient plant may create a progeny plant with combined desirable loci which may provide quantitatively additive effect of a particular characteristic. In that case, QTL mapping can be involved to facilitate the breeding process.

A QTL (quantitative trait locus) mapping can be applied to determine the parts of the donor plant's genome conferring the desirable phenotype, and facilitate the breeding methods. Inheritance of quantitative traits or polygenic inheritance refers to the inheritance of a phenotypic characteristic that varies in degree and can be attributed to the interactions between two or more genes and their environment. Though not necessarily genes themselves, quantitative trait loci (QTLs) are stretches of DNA that are closely linked to the genes that underlie the trait in question. QTLs can be molecularly identified to help map regions of the genome that contain genes involved in specifying a quantitative trait. This can be an early step in identifying and sequencing these genes.

Typically, QTLs underlie continuous traits (those traits that vary continuously, e.g. yield, height, level of resistance to virus, etc.) as opposed to discrete traits (traits that have two or several character values, e.g. smooth vs. wrinkled peas used by Mendel in his experiments). Moreover, a single phenotypic trait is usually determined by many genes. Consequently, many QTLs are associated with a single trait.

A quantitative trait locus (QTL) is a region of DNA that is associated with a particular phenotypic trait. Knowing the number of QTLs that explains variation in the phenotypic trait tells about the genetic architecture of a trait. It may tell that a trait is controlled by many genes of small effect, or by a few genes of large effect or by a several genes of small effect and few genes of larger effect.

Another use of QTLs is to identify candidate genes underlying a trait. Once a region of DNA is identified as contributing to a phenotype, it can be sequenced. The DNA sequence of any genes in this region can then be compared to a database of DNA for genes whose function is already known.

In a recent development, classical QM analyses are combined with gene expression profiling i.e. by DNA microarrays. Such expression QTLs (e-QTLs) describes cis- and trans-controlling elements for the expression of often disease-associated genes. Observed epistatic effects have been found beneficial to identify the gene responsible by a cross-validation of genes within the interacting loci with metabolic pathway- and scientific literature databases.

QTL mapping is the statistical study of the alleles that occur in a locus and the phenotypes (physical forms or traits) that they produce (see, Meksem and Kahl, The handbook of plant genome mapping: genetic and physical mapping, 2005, Wiley-VCH, ISBN 3527311165, 9783527311163). Because most traits of interest are governed by more than one gene, defining and studying the entire locus of genes related to a trait gives hope of understanding what effect the genotype of an individual might have in the real world.

Statistical analysis is required to demonstrate that different genes interact with one another and to determine whether they produce a significant effect on the phenotype. QTLs identify a particular region of the genome as containing one or several genes, i.e. a cluster of genes that is associated with the trait being assayed or measured. They are shown as intervals across a chromosome, where the probability of association is plotted for each marker used in the mapping experiment.

To begin, a set of genetic markers must be developed for the species in question. A marker is an identifiable region of variable DNA. Biologists are interested in understanding the genetic basis of phenotypes (physical traits). The aim is to find a marker that is significantly more likely to co-occur with the trait than expected by chance, that is, a marker that has a statistical association with the trait. Ideally, they would be able to find the specific gene or genes in question, but this is a long and difficult undertaking. Instead, they can more readily find regions of DNA that are very close to the genes in question. When a QTL is found, it is often not the actual gene underlying the phenotypic trait, but rather a region of DNA that is closely linked with the gene.

For organisms whose genomes are known, one might now try to exclude genes in the identified region whose function is known with some certainty not to be connected with the trait in question. If the genome is not available, it may be an option to sequence the identified region and determine the putative functions of genes by their similarity to genes with known function, usually in other genomes. This can be done using BLAST, an online tool that allows users to enter a primary sequence and search for similar sequences within the BLAST database of genes from various organisms.

Another interest of statistical geneticists using QTL mapping is to determine the complexity of the genetic architecture underlying a phenotypic trait. For example, they may be interested in knowing whether a phenotype is shaped by many independent loci, or by a few loci, and how do those loci interact. This can provide information on how the phenotype may be evolving.

Molecular markers are used for the visualization of differences in nucleic acid sequences. This visualization is possible due to DNA-DNA hybridization techniques (RFLP) and/or due to techniques using the polymerase chain reaction (e.g. STS, SNPs, microsatellites, AFLP). All differences between two parental genotypes will segregate in a mapping population based on the cross of these parental genotypes. The segregation of the different markers may be compared and recombination frequencies can be calculated. The recombination frequencies of molecular markers on different chromosomes are generally 50%. Between molecular markers located on the same chromosome the recombination frequency depends on the distance between the markers. A low recombination frequency usually corresponds to a low distance between markers on a chromosome. Comparing all recombination frequencies will result in the most logical order of the molecular markers on the chromosomes. This most logical order can be depicted in a linkage map (Paterson, 1996, Genome Mapping in Plants. R.G. Landes, Austin.). A group of adjacent or contiguous markers on the linkage map that is associated to a reduced disease incidence and/or a reduced lesion growth rate pinpoints the position of a QTL.

The nucleic acid sequence of a QTL may be determined by methods known to the skilled person. For instance, a nucleic acid sequence comprising said QTL, or a resistance-conferring part thereof may be isolated from a donor plant by fragmenting the genome of said plant and selecting those fragments harboring one or more markers indicative of said QTL. Subsequently, or alternatively, the marker sequences (or parts thereof) indicative of said QTL may be used as (PCR) amplification primers, in order to amplify a nucleic acid sequence comprising said QTL from a genomic nucleic acid sample or a genome fragment obtained from said plant. The amplified sequence may then be purified in order to obtain the isolated QTL. The nucleotide sequence of the QTL, and/or of any additional markers comprised therein, may then be obtained by standard sequencing methods.

One or more such QTLs associated with a desirable trait in a donor plant can be transferred to a recipient plant to incorporate the desirable trait into progeny plants by transferring and/or breeding methods.

In one embodiment, an advanced backcross QTL analysis (AB-QTL) is used to discover the nucleotide sequence or the QTLs responsible for the resistance of a plant. Such method was proposed by Tanksley and Nelson in 1996 (Tanksley and Nelson, 1996, Advanced backcross QTL analysis: a method for simultaneous discovery and transfer of valuable QTL from un-adapted germplasm into elite breeding lines. Theor Appl Genet 92:191-203) as a new breeding method that integrates the process of QTL discovery with variety development, by simultaneously identifying and transferring useful QTL alleles from un-adapted (e.g., land races, wild species) to elite germplasm, thus broadening the genetic diversity available for breeding. AB-QTL strategy was initially developed and tested in tomato, and has been adapted for use in other crops including rice, maize, wheat, pepper, barley, and bean. Once favorable QTL alleles are detected, only a few additional marker-assisted generations are required to generate near isogenic lines (Nits) or introgression lines (ILs) that can be field tested in order to confirm the QTL effect and subsequently used for variety development.

Isogenic lines in which favorable QTL alleles have been fixed can be generated by systematic backcrossing and introgressing of marker-defined donor segments in the recurrent parent background. These isogenic lines are referred to as near isogenic lines (NILs), introgression lines (Its), backcross inbred lines (BILs), backcross recombinant inbred lines (BCRIL), recombinant chromosome substitution lines (RCSLs), chromosome segment substitution lines (CSSLs), and stepped aligned inbred recombinant strains (STAIRSs). An introgression line in plant molecular biology is a line of a crop species that contains genetic material derived from a similar species. ILs represent NILs with relatively large average introgression length, while BILs and BCRILs are backcross populations generally containing multiple donor introgressions per line. As used herein, the term “introgression lines or ILs” refers to plant lines containing a single marker defined homozygous donor segment, and the term “pre-ILs” refers to lines which still contain multiple homozygous and/or heterozygous donor segments.

To enhance the rate of progress of introgressi on breeding, a genetic infrastructure of exotic libraries can be developed. Such an exotic library comprises a set of introgression lines, each of which has a single, possibly homozygous, marker-defined chromosomal segment that originates from a donor exotic parent, in an otherwise homogenous elite genetic background, so that the entire donor genome would be represented in a set of introgression lines. A collection of such introgression lines is referred as libraries of introgression lines or IL libraries (ILLs). The lines of an ILL cover usually the complete genome of the donor, or the part of interest. Introgression lines allow the study of quantitative trait loci, but also the creation of new varieties by introducing exotic traits. High resolution mapping of QTL using ILLs enable breeders to assess whether the effect on the phenotype is due to a single QTL, or to several tightly linked QTL affecting the same trait. In addition, sub-ILs can be developed to discover molecular markers which are more tightly linked to the QTL of interest, which can be used for marker-assisted breeding (MAB). Multiple introgression lines can be developed when the introgression of a single QTL is not sufficient to result in a substantial improvement in agriculturally important traits (Gur and Zatnir, Unused natural variation can lift yield barriers in plant breeding, 2004, PLoS Biol.; 2(1.0):e245).

### Tissue Culture

As used herein, the term “tissue culture” indicates a composition comprising isolated cells of the same or a different type or a collection of such cells organized into parts of a plant.

Exemplary types of tissue cultures are protoplasts, calli, plant clumps, and plant cells that can generate tissue culture that are intact in plants or parts of plants, such as embryos, pollen, flowers, seeds, leaves, stems, roots, root tips, anthers, pistils, meristematic cells, axillary buds, ovaries, seed coat, endosperm, hypocotyls, cotyledons and the like. Means for preparing and maintaining plant tissue culture are well known in the art. By way of example, a tissue culture comprising organs has been used to produce regenerated plants. U.S. Pat. Nos. 5,959,185, 5,973,234, and 5,977,445 describe certain techniques, the disclosures of which are incorporated herein by reference. See also, e.g., Vinay and Afrox, Plant Tissue Culture, 2015, I. K, International Publishing House; Kavyashree and Gayatri, Plant Tissue Culture, 2015, Alpha Science Intl Ltd.; and Michael A. Dirr, The Reference Manual of Woody Plant Propagation: From Seed to Tissue Culture, Second Edition, 2006, Timber Press.

Tissue culture of Justicia can be used for the in vitro regeneration of Justicia plants. Standard plant tissue cultures methods and regeneration of plants therefrom are well known in the art. Thus, another aspect of this disclosure is to provide cells which upon growth and differentiation produce Justicia plants. In some embodiments, such tissue culture methods can be used to produce regenerated plants from cells and tissues of the ‘Befu’ cultivar, wherein such regenerated plants have all of the physiological and morphological characteristics of ‘Befu.’

### Tea and Tea-Like Beverages

In some embodiments, the present disclosure teaches teas or tea-type beverages produced from Justicia plants. In some embodiments, the present disclosure teaches teas or tea-type beverages produced from Justicia sanguinis plants. In some embodiments, the present disclosure teaches teas or tea-type beverages produced from Justicia sanguinis plant named ‘Befu.’

As used herein, a “tea” or “tea-type beverage” refer generally to any drink made by infusing plant parts in water. Typically, the infusion takes place in hot, very hot or boiling water, which may be consumed hot, warm, at room temperature, chilled or cold. Generally, a tea is made by infusing the fresh or dried, whole or crushed leaves of the plant in boiling water. A. tea or tea-type beverage, also known as “infusions” or “tisanes,” can easily be made from herbs, medicinal plants or tea plants (Camellia sinensis) by putting all or parts of the fruits, herbs, medicinal plants, or tea (such as, for example, in the form of leaves or powder) in a cup of hot or boiling water. For some teas, such as fruit teas or teas made from herbs or medicinal plants, the steep time is rather long, whereas for various kinds of tea plants, maintaining a certain steep time is required for producing the best flavor. The flavor and taste can depend greatly depends on water quality and temperature.

Tea is generally prepared as green leaf tea or black leaf tea. The method of preparing such teas is well known to those skilled in the art. Generally, to prepare black leaf tea, fresh green leaves of a plant are subjected to mild drying, comminuted, fermented (in which enzymes in the leaf tea oxidize various substrates to produce brown-colored products) and then fired (to dry the tea leaves). In some embodiments, no fermentation process is used to produce the tea.

Green leaf tea is not exposed to the fermentation process. Partial fermentation may be used to produce intermediate-type teas known as “oolong” tea.

In some embodiments, tea based beverages can be prepared by methods other than infusing leaves in hot water and served in ways other than poured from tea pots. For example they can be made with concentrates or powders that are mixed with hot water in vending machines or used to prepare ready to drink teas in cans and bottles. Some tea products involve accelerated infusion, enhanced colors, and added aromas.

For examples and descriptions of teas and the processes to make teas, see, e.g., U.S. Published Patent Application Nos. 2014/0295049, 2008/0095913 and 2008/0107774; and, Keating and Long, How to Make Tea: The Science Behind the Leaf (How to Make Series), 2015, Ivy Press.

### EXAMPLES

The foregoing examples of the related art and limitations related therewith are intended to be illustrative and not exclusive. Other limitations of the related art will become apparent to those of skill in the art upon a reading of the specification.

### Example 1—Discovery of ‘Befu’ in a Cultivated Area

The plants of the present invention were discovered growing in a cultivated area on private land in Orlando, Fla., U.S.A. The parentage of the discovered plants is unknown. Possible unconfirmed origin of original plants grown in this cultivated area may have been from the Cameroon.

### Example 2—Asexual Reproduction of ‘Befu’

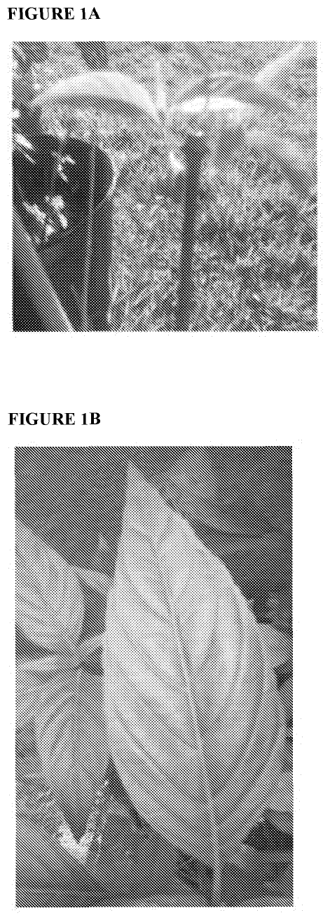

Plants of the present invention were asexually reproduced via stein cuttings in Orlando, Fla., U.S.A. See FIG. 1A.

### Example 3—Botanical Description of ‘Befu’

The following is a detailed description of the new Justicia cultivar named ‘Befu’. Data was collected in Orlando, Fla., U.S.A.

‘Befu’ is an herb which grows to about 40 inches in maximum height with leaves opposite. See FIG. 1B.

The plant is green with red rings present at the base of petiole. To date no flower structures have been observed on plants of ‘Befu.’

Color determinations are in accordance with The Royal Horticultural Society Colour Chart 2001 edition, except where general color terms of ordinary dictionary significance are used. The growing requirements are similar to those typically used for this genus of plants. ‘Befu’ has not been tested under all possible conditions and phenotypic differences may be observed with variations in environmental, climatic, and cultural conditions, however, without any variance in genotype.

The botanical classification is proposed to be Justicia sanguinis

Disease and pest resistance: Plants of the new cultivar have not been observed for disease and pest resistance.

The following traits in combination distinguish the Justicia sanguinis ‘Befu’ from a check variety Justicia Plant named ‘ZEBRA’.

TABLE 1
Justicia sanguinis ‘Beth’ Plant Traits

| Characteristics | New Variety (Befu) | Check Variety (Zebra) |
|---|---|---|
| Plant growth habit | Upright | Upright |
| Plant propagation | Asexually propagated by stem | Terminal cuttings |
| | cuttings and cloning | |
| Plant vigor | N/A | Medium |
| Height | Up to 101.6 cm | 23.2 cm |
| Leaf arrangement | Opposite | Opposite |
| Compound or | Single | Single |
| single | | |
| Leaf shape | Acute, Lanceolate | Cordate |
| Leaf apex | Accuminate | Apiculate |
| Leaf base | Obtuse | Cordate |
| Leaf margin | Crenate | Entire |
| Venation pattern | Pinnate | Pinnate |
| Leaf attachment | Petiolate | Petiolate |
| Resistance to pests | Plants have not been observed | Plants have not been observed |
| or diseases | for disease and pest resistance | for disease and pest resistance |
| Genetically- | NO | NO |
| modified organism | | |
| Hemoglobin | High | N/A |

### Example 4—Morphological Comparisons of Justicia sauguinis ‘Befu’ Plant with other Justicia Species

Applicant will conduct further morphological comparisons between the presently disclosed Justicia sanguinis species of plants, and other plants in the Justicia genus. The morphological features of the new species of Justicia sanguinis plant, named ‘Befu’ will be compared with one or more commonly known Justicia plant species selected from the group consisting of Justicia Americana, Justicia hrandegeeana, Justicia carnea, Justicia ovata, Justicia procumbens, Justicia pectoralis Jacq., Justicia gendarussa Buim. f., Justicia anselliana, Justicia adhatoda, Justicia secunda and Justicia picnfolia.

A list of the various morphologies that will be compared between the various species includes, but is not limited to, botanical classification, plant life forms, plant growth habit, plant origin, plant propagation, height, width, vigor, time to initiate roots, time to produce a rooted cutting or linger, time to harvest, growth rate, root system, stem features (branching habit, average number of main stems, pinching, stem diameter, stern length, stern branch strength, stem color, stem shape, pubescence, internode length, aspect, strength), foliage features (texture, leaf arrangement, compound or single, quantity of leaves per stern, leaf shape, leaf apex, leaf base, leaf length, leaf width, pubescence, leaf margin, young leaf color (lower and upper surface), mature leaf color (lower and upper surface), vein color, venation pattern, leaf attachment, petiole dimensions, petiole color), flower features (inflorescence arrangement, flowering habit, quantity of flowers per stem, quantity of flower buds per stem, quantity of flowers and buds per plant, natural flowering season, fragrance, flower bud length, flower bud diameter, flower bud shape, bud color, rate of bud opening, flower aspect, flower shape, flower dimension, flower longevity, petal appearance, petal texture, number of petals, fused or unfused, petal appearance, petal shape, petal margin, petal apex, petal length, petal width, petal color), sepal features (number of sepal, sepal aspect, sepal shape, sepal margin sepal apex, sepal base, sepal surface, sepal dimensions, young sepal color, mature sepal color), calyx shape, calyx dimension, peduncle dimensions, peduncle aspect, peduncle color, peduncle strength, and reproductive organ features (stamen number, anther shape, anther dimensions, anther color, amount of pollen, pollen color, pistil number, pistil dimensions, stigma shape, stigma color, style length, style color, ovary color).

The cultivated ‘Befu’ cultivar will also he compared to other Justicia plants found near the cultivated space where the ‘Befu’ was identified. The morphological comparison will include a comparison of one or more of the features described in the preceding paragraph. It is expected that this data will further demonstrate the morphological differences between the presently disclosed Justicia sanguinis species, with other existing Justicia species.

### Example 5—Tea Beverage Made From ‘Befu’

Plant parts were placed into warm water to make a tea drink which was consumed. Consumption of the tea produces a general, overall feeling of improved well-being and healthfulness. Tea from the presently disclosed ‘Befu’ plant have been produced in a range of temperatures ranging from slightly above freezing to boiling temperatures.

### Example 6—Comparisons of Extracts from Justicia sanguinis ‘Befu’ Plant with Extracts from Other Justicia Species

Applicant has hereby described extracts and methods of producing the same, of a newly discovered Justicia sanguinis ‘Befu’ plant with unique properties and applications. Applicant has demonstrated through DNA and morphological analysis that the presently disclosed ‘Befu’ plant represents a previously unknown species of Justicia. In order to further distinguish the presently claimed extracts produced from Justicia sanguinis from those of other plants, Applicant will compare the claimed extracts with those produced from other Justicia species.

Extracts will be produced as described in earlier portions of this disclosure. Briefly, plant leaf tissue from each plant will be added to water at a temperature of 180° F. a water to leaf ratio of 1:1 to 30:1. The liquid portion of the extract will be removed and analyzed via ICP-MS. Extracts from Justicia sanguinis plant, named ‘Befu’ will be compared with the extracts from one or more commonly known Justicia plant species selected from the group consisting of Justicia Americana, Justicia brandegeeana, Justicia carnea, Justicia ovata, Justicia procumbens, Justicia pectoralis Jacq., Justicia gendarussa Buim. f., Justicia anselliana, Justicia adhatoda, Justicia secunda and Justicia pictifolia.

Extracts form the cultivated ‘Befu’ cultivar will also be compared to extracts from other Justicia plants found near the cultivated space where the ‘Befu’ was identified. The morphological comparison will include a comparison of one or more of the features described in the preceding paragraph. It is expected that this data will further demonstrate the morphological differences between the presently disclosed Justicia sanguinis species, with other existing Justicia species.

### Example 7—Identification of New Justicia Species via DNA Analysis

A tissue sample, consisting of photosynthetic leaf material, of the plant of the present disclosure was preserved by silica gel desiccation. A voucher specimen (see voucher data below) to document the plant from which the sample was taken was collected, dried, and deposited in the US National Herbarium (Smithsonian Institution).

Voucher Specimen: Justicia sp. (Acanthaceae). Herb to 40 cm in height, leaves opposite, green with red ring at base of petiole, and no Flowers. DNA barcode voucher was taken from plant in cultivation in Orlando, Fla.; possible but not confirmed origin in Cameroon.

DNA was extracted from the silica-dried sample using a CT AB extraction method and stored at 80° C. Routine PCR was employed and primers for each marker followed Kress et al. (2010). Cycle sequencing protocols were the same for all markers. Following cycle sequencing, products are purified on a column of sephadex G50 in Millipore Multi-Screen 96-well plates and. sequence reactions read on an ABI 3730. Forward and reverse sequences were assembled and aligned using Geneious Pro 4.6, TRANSALIGN, and Muscle depending on the DNA barcode marker. All DNA barcode sequences have been submitted to GenBank. DNA sequences from the unknown plant sample were compared against the plant DNA sequence data assembled in GenBank using the BLASTn algorithm (the core GenBank search engine) and default search parameters. In addition, the voucher specimen was compared to reference collections in the United States National Herbarium to confirm the DNA barcode identification.

To establish identity of the plant, DNA from photosynthetic leaf material of the plant was employed for DNA barcoding. DNA barcodes (including the markers rbcL, matK, and trnH-psbA) was generated by the protocol outlined by Kress et al. (2009, 2010) and Kress and Erickson (2012). BLAST results from the DNA barcode marker comparisons to GenBank sequence data established the plant to be of the genus Justicia in the family Acanthanceae. The DNA barcode sequence data however, were not able to identify a species for the plant, suggesting that the sample belonged to a new species of Justicia. The generic identity of the sample was further confirmed by a taxonomic specialist in the Department of Botany at the United States National Herbarium. Justicia includes over 600 species that are found in pantropical regions. These species are known to be evergreen perennials and shrubs with leaves that are characteristically petiolate, strongly veined, and with a margin that is usually entire (FIG. 11B). Based on these results, the ‘Befu’ plant was assigned to a new species named Justicia sanguinis.

### Deposit Information

A voucher specimen of ‘Befu’ has been deposited in the U.S. Herbarium (Smithsonian Institution). DNA barcode voucher sent by Wilfred F. Ngwa taken from a plant cultivation in Orlando, Fla., U.S.A. Dated: 19 Jun. 2017. Verification: W. J. Kress #17-8936 (USA).

In addition, a sample of the ‘Befu’ seed and/or of this disclosure has been or will be deposited with [a Depositary Institution Having Acquired the Status of International Depositary Authority Under the Budapest Treaty].

To satisfy the enablement requirements of 35 U.S.C. 112, and to certify that the deposit of the isolated strain of the present disclosure meets the criteria set forth in 37 C.F.R. 1,801-1.809, Applicants hereby make the following statements regarding the deposited ‘Befu’ (deposited as XXXX Accession No. ______):
1. During the pendency of this application, access to the disclosure will be afforded to the Commissioner upon request;
2. All restrictions on availability to the public will be irrevocably removed upon granting of the patent under conditions specified in 37 CFR 1.808;
3. The deposit will be maintained in a public repository for a period of 30 years or 5 years after the last request or for the effective life of the patent, whichever is longer;
4. A test of the viability of the biological material at the time of deposit will be conducted by the public depository under 37 CFR. 1.807; and
5. The deposit will be replaced if it should ever become unavailable.

Access to this deposit will be available during the pendency of this application to persons determined by the Commissioner of Patents and Trademarks to be entitled thereto under 37 C.F.R. § 1.14 and 35 U.S.C. § 122. Upon allowance of any claims in this application, all restrictions on the availability to the public of the variety will be irrevocably removed by affording access to a deposit of [at least XXX seeds] of the same variety with the XXXX deposit.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the non-limiting exemplary methods and materials are described herein.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. Nothing herein is to be construed as an admission that the present invention is not entitled to antedate such publication by virtue of prior invention.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### Numbered Embodiments of the Disclosure

Notwithstanding the appended claims, the disclosure sets forth the following numbered embodiments:
1. A Justicia sanguinis plant named ‘Befu’, or a plant part thereof, or a plant cell thereof, wherein a representative sample of seed or tissue culture of said Justicia sanguinis plant has been deposited with XXXX under XXXX No. ______.

2. The Justicia sanguinis plant part of embodiment 1, wherein the Justicia sanguinis plant part is a leaf or a stem.

3. A Justicia sanguinis plant having all of the characteristics of the Justicia sanguinis plant named ‘Befu’ listed in Table 1 when grown under the same environmental conditions, or a plant part or a plant cell thereof.

4. A Justicia sanguinis plant, or a plant part thereof, having all of the physiological and morphological characteristics of the Justicia sanguinis plant of any one of embodiments 1, 2 or 3.

5. A tissue culture of regenerable cells produced from the plant, plant part or plant cell of any one of embodiments 1, 2, 3, or 4, wherein a new plant regenerated from the tissue culture has all of the characteristics of Justicia sanguinis plant named ‘Befu’ listed in Table 1 when grown under the same environmental conditions.

6. A Justicia sanguinis plant regenerated from the tissue culture of embodiment 5, said plant having all the characteristics of Justicia sanguinis of any one of embodiments 1, 2, 3, or 4.

7. A Justicia sanguinis leaf produced from the Justicia sanguinis plant of any one of embodiments 1, 3, 4, or 6.

8. A method for producing a Justicia sanguinis leaf comprising a) growing the Justicia sanguinis plant of any one of embodiments 1, 2, 3, 4 or 6 to produce a Justicia sanguinis leaf, and b) harvesting said Justicia sanguinis leaf.
9. A Justicia Justicia leaf produced by the method of embodiment 8.
10. A method for producing a Justicia sanguinis seed comprising crossing the Justicia sanguinis plant of embodiment 1, 2, 3, 4, or 6 with itself or a second, distinct plant.
11. An F1 Justicia sanguinis seed produced by the method of embodiment 10.
12. A method for producing a Justicia sanguinis seed comprising self-pollinating the Justicia sanguinis plant of embodiment 1, 2, 3, 4, or 6 and harvesting the resultant Justicia sanguinis seed.
13. A Justicia sanguinis seed produced by he method of embodiment 12.
14. A method of producing a Justicia sanguinis plant derived from the Justicia sanguinis named ‘Befu’, the method comprising (a) crossing the plant of embodiment 1, 2, 3, 4, or 6 with a second plant to produce a progeny plant.
15. The method of embodiment 14 further comprising the step of:
   (b) crossing the progeny plant derived from Justicia sanguinis plant with itself or a second plant to produce a seed of progeny plant of subsequent generation;
   (c) growing the progeny plant of the subsequent generation from the seed
   (d) crossing the progeny plant of the subsequent generation with itself or a second plant, to produce a Justicia sanguinis plant derived from the Justicia sanguinis plant.
16. The method of embodiment 15 further comprising the step of: (e) repeating steps (b) and/or (c) to produce a Justicia sanguinis plant derived from the Justicia sanguines plant of any one of embodiments 1, 2, 3, 4, or 6.
17. The plant of embodiment 1, 2, 3, 4, or 6 comprising a single locus conversion and otherwise essentially all the characteristics of the Justicia sanguinis plant of any one of embodiments 1, 2, 3, 4 or 6 when grown in the same environmental conditions.
18. The plant of embodiment 17 wherein the single locus conversion confers said plant with herbicide resistance.
19. The plant of embodiment 17 wherein the single locus conversion is an artificially mutated gene or nucleotide sequence.
20. The plant of embodiment 17 wherein the single locus conversion is a gene that has been modified through the use of new breeding techniques.
21. A method of introducing a desired trait into Justicia sanguinis plant comprising:
   (a) crossing a first Justcia sanguinis plant of any one of embodiments 1, 2, 4, 5 or 6 with a second Justicia plant that comprises a desired trait to produce F1 progeny plants.
22. The method of embodiment 21, further comprising the steps of:
   (b) selecting one or more progeny plants that have the desired trait to produce selected progeny plants;
   (c) crossing the selected progeny plants with the first Justicia sanguinis plant so as to produce backcross progeny plants;
   (d) selecting for backcross progeny plants that have the desired trait and all of the physiological and morphological characteristics of the first Justicia sanguinis plant when grown in the same environmental conditions to produce selected backcross progeny plants; and
   (e) repeating steps (c) and (d) three or more times in succession to produce selected fourth or higher backcross progeny plants that comprise the desired trait and all of the physiological and morphological characteristics of the first Justicia sanguinis plant when grown in the same environmental conditions.
23. A beverage comprising an extract of the plant or plant part of any one of embodiments 1-7, 9, and 17-20.
23.1 The beverage of embodiment 23, wherein the plant part is a leaf, or portion thereof.
24. A tea comprising an extract of the plant or plant part of any one of embodiments 1-7, 9, and 17-20.
24.1 The tea of embodiment 24, wherein the plant part is a leaf, or portion
25. An edible composition comprising an extract of the plant or plant part of any one of embodiments 1-7, 9, and 17-20.
25.1 The edible composition of embodiment 25, wherein the plant part is a leaf, or portion
26. A method of preparing a beverage comprising placing the plant part of any one or more of embodiments 1-7, 9, and 17-20 in contact with a solvent.
27. The method of embodiment 26, wherein the plant part is a leaf or a portion of a leaf.
28. The method of embodiment 27, wherein the leaf or portion of a leaf is partially or completely dried before placing it in the liquid.
29. The method of embodiment 26, wherein the solvent is water.
30. The method of any one of embodiments 26-29, wherein the solvent is warm, hot or boiling when the leaf or portion of a leaf is placed into the solvent.
31. The method of any one of embodiments 26-29, wherein the solvent is between 80 and 230 degrees Farenheit
32. A new and distinct species of Justicia sanguinis plants as described and illustrated.
33. A new and distinct variety Justicia sanguinis named ‘Befu’ as described and illustrated.
34. A plant cell from a Justicia sanguinis plant or an asexual clone thereof.
35. A plant cell from a Justicia plant or an asexual clone thereof, wherein a representative sample of seed or tissue culture of said Justicia plant has been deposited with XXXX under XXXX No. ______.
35.1 The plant cell of embodiments 34 or 35, wherein the Justicia plant is the variety ‘Befu.’
35.2 The plant cell of any one of embodiments 34-35.1, wherein the Justicia plant has all of the characteristics of the Justicia ‘Befu’ plant listed in Table 1 when grown under the same environmental conditions.
35.3 The plant cell of any one of embodiments 34-35.2, wherein said Justicia plant is regenerated from a seed or tissue culture deposited with XXXX under XXXX No. ______.
35.4 The plant cell of any one of embodiments 34-35.3, wherein said Justicia plant is obtainable from a seed or tissue culture deposited with XXXX under XXXX No. ______.
36. Use of a first Justicia plant, wherein the first Justicia plant comprises the plant cell of any one of embodiments 34-35.4, for crossing with itself or with a second Justicia plant to produce an F1 seed; wherein the F1 seed produces and F1 plant.
37. Use of a seed, cutting or plant cell from a first Justicia plant comprising the plant cell of any one of embodiments 34-35.4 to produce a second Justicia plant.
38. A non-viable edible product comprising an extract of a Justicia plant, a plant part thereof or an asexual clone thereof, comprising the plant cell according to any one of embodiments 34-35.4.
39. A dry, non-viable plant part from a Justicia sanguinis plant.
39.1 The dry, non-viable plant part of embodiment 39, wherein a representative sample of seed or tissue culture of said Justicia plant has been deposited with XXXX under XXXX No. ______.
39.2 The dry, non-viable plant part of any one of embodiments 39-39.1, wherein the Justicia plant is the variety ‘Befu.’
39.3 The dry, non-viable plant part of any one of embodiments 39-39.2, wherein the Justicia plant has all of the characteristics of the Justicia ‘Befu” plant listed in Table 1 when grown under the same environmental conditions.
39.4 The dry, non-viable plant part of any one of embodiments 39-39.3, wherein said Justicia plant is regenerated from a seed or tissue culture deposited with XXXX under XXXX No. ______.
39.5 The dry, non-viable plant part of any one of embodiments 39-39.4, wherein said Justicia plant is obtainable from a seed or tissue culture deposited with XXXX under XXXX No. ______.
40. An assemblage of dry, non-viable tissue from a Justicia plant, a plant part thereof or an asexual clone thereof, comprising the plant cell according to any one of embodiments 34-35.4.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

What is claimed is:

1. A Justicia sanguinis plant named ‘Befu’, or a plant part thereof, or a plant cell thereof, wherein a representative sample of seed or tissue culture of said Justicia sanguinis plant has been deposited with XXXX under XXXX No. ______.

2. The Justicia sanguinis plant part of claim 1, wherein the Justicia sanguinis plant part is a leaf or a stem.

3. A Justicia sanguinis plant having all of the characteristics of the Justicia sanguinis plant named ‘Befu’ listed in Table 1 when grown under the same environmental conditions, or a plant part or a plant cell thereof.

4. A Justicia sanguinis plant, or a plant part thereof, having all of the physiological and morphological characteristics of the Justicia sanguinis plant of any one of claim 1, 2 or 3.

5. A tissue culture of regenerable cells produced from the plant, plant part or plant cell of any one of claim 1, 2, 3, or 4, wherein a new plant regenerated from the tissue culture has all of the characteristics of Justicia sanguinis plant named ‘Befu’ listed in Table 1 when grown under the same environmental conditions.

6. A Justicia sanguinis plant regenerated from the tissue culture of claim 5, said plant having all the characteristics of Justicia sanguinis of any one of claim 1, 2, 3, or 4.

7. A Justicia sanguinis leaf produced from the Justicia sanguinis plant of any one of claim 1, 3, 4, or 6.

8. A method for producing a Justicia sanguinis leaf comprising a) growing the Justicia sanguinis plant of any one of claim 1, 3, 4 or 6 to produce a Justicia sanguinis leaf, and b) harvesting said Justicia sanguinis leaf.

9. A Justicia Justicia leaf produced by the method of claim 8.

10. A method for producing a Justicia sanguinis seed comprising crossing the Justicia sanguinis plant of claim 1, 2, 3, 4, or 6 with itself or a second, distinct plant.

11. An F 1 Justicia sanguinis seed produced by the method of claim 10.

12. A method for producing a Justicia sanguinis seed comprising self-pollinating the Justicia sanguinis plant of claim 1, 2, 3, 4, or 6 and harvesting the resultant Justicia sanguinis seed.

13. A Justicia sanguinis seed produced by the method of claim 12.

14. A method of producing a Justicia sanguines plant derived from the Justicia sanguines named ‘Befu’, the method comprising (a) crossing the plant of claim 1, 2, 3, 4, or 6 with a second plant to produce a progeny plant.

15. The method of claim 14 further comprising the step of:
(b) crossing the progeny plant derived from Justicia sanguinis plant with itself or a second plant to produce a seed of progeny plant of subsequent generation;
(c) growing the progeny plant of the subsequent generation from the seed
(d) crossing the progeny plant of the subsequent generation with itself or a second plant, to produce a Justicia sanguinis plant derived from the Justicia sanguinis plant.

16. The method of claim 15 further comprising the step of: (e) repeating steps (b) and/or (c) to produce a Justicia sanguinis plant derived from the Justicia sanguinis plant of any one of claim 1, 2, 3, 4, or 6.

17. The plant of claim 1, 2, 3, 4, or 6 comprising a single locus conversion and otherwise essentially all the characteristics of the Justicia sanguinis plant of any one of claim 1, 2, 3, 4 or 6 when grown in the same environmental conditions.

18. The plant of claim 17 wherein the single locus conversion confers said plant with herbicide resistance.

19. The plant of claim 17 wherein the single locus conversion is an artificially mutated gene or nucleotide sequence.

20. The plant of claim 17 wherein the single locus conversion is a gene that has been modified through the use of new breeding techniques.

21. A method of introducing a desired trait into Justicia sanguinis plant comprising:
(a) crossing a first Justicia sanguinis plant of any one of claim 1, 2, 4, 5 or 6 with a second Justicia plant that comprises a desired trait to produce F1 progeny plants.

22. The method of claim 21, further comprising the steps of:
(b) selecting one or more progeny plants that have the desired trait to produce selected progeny plants;
(c) crossing the selected progeny plants with the first Justicia sanguinis plant so as to produce backcross progeny plants;
(d) selecting for backcross progeny plants that have the desired trait and all of the physiological and morphological characteristics of the first Justicia sanguinis plant when grown in the same environmental conditions to produce selected backcross progeny plants; and
(e) repeating steps (c) and (d) three or more times in succession to produce selected fourth or higher backcross progeny plants that comprise the desired trait and all of the physiological and morphological characteristics of the first Justicia sanguinis plant when grown in the same environmental conditions.

23. A beverage comprising an extract of the plant or plant part of any one of claims 1-7, 9, and 17-20.

24. A tea comprising an extract of the plant or plant part of any one of claims 1-7, 9, and 17-20.

25. An edible composition comprising an extract of the plant or plant part of any one of claims 1-7, 9, and 17-20.

26. A method of preparing a beverage comprising placing the plant part of any one or more of claims 1-7, 9, and 17-20 in contact with a solvent.

27. The method of claim 26, wherein the plant part is a leaf or a portion of a leaf.

28. The method of claim 27, wherein the leaf or portion of a leaf is partially or completely dried before placing it in the liquid.

29. The method of claim 26, wherein the solvent is water.

30. The method of any one of claims 26-29, wherein the solvent is warm, hot or boiling when the leaf or portion of a leaf is placed into the solvent.

31. The method of any one of claims 26-29, wherein the solvent is between 80 and 230 degrees Fahrenheit.

32. A new and distinct species of Justicia sanguinis plants as described and illustrated.

33. A new and distinct variety of Justicia sanguinis named ‘Befu’ as described and illustrated.
